(19) United States
(12) Patent Application Publication
(10) Pub. No.: US 2011/0021974 A1
(43) Pub. Date: Jan. 27, 2011

(54) RETINITIS PIGMENTOSA TREATMENT AND PROPHALAXIS

(76) Inventors: Totada R. Shantha, McDonough, GA (US); Jessica Shantha, McDonough, GA (US); Erica Maya Shantha, McDonough, GA (US)

Correspondence Address: PATWRITE LLC, 408 W. MAIN ST., MARSHALLTOWN, IA, 50158-5759, US

(21) Appl. No.: 12/898,524

(22) Filed: Oct. 5, 2010

(51) Int. Cl. A61F 9/00; A61K 38/28; A61K 38/30; A61K 33/00; A61K 38/19; A61K 39/395; A61K 9/127; A61P 27/02; A61P 29/00; A61P 31/12; A61P 31/00; A61P 9/00

(52) U.S. Cl. 604/20; 604/22; 514/5.9; 514/6.5; 514/8.6; 424/600; 424/85.1; 424/133.1; 424/178.1; 424/142.1; 424/450

(57) ABSTRACT

The invention relates to a method of instilling insulin ophthalmic drops in the conjunctival sac for treating retinitis pigmentosa due to any etiological factors both genetic and non genetic. The retinitis pigmentosa is treated with Insulin and/or IGF-I with or without known anti-retinitis pigmentosa therapeutic, pharmaceutical, biochemical, and biological agents or compounds. The invention furthermore uses this method as prophylactic on patients where the patients are predisposed to develop retinitis pigmentosa. The invention additionally treats other oculopathies associated with and/or contributing to retinitis pigmentosa.

### FIELD OF THE INVENTION

The invention relates to the therapeutic agents and methods for treating retinitis pigmentosa in humans and animals. The inventive method described here can be used on patients with other known therapeutic, pharmaceutical, biochemical, nurticeuticals and biological agents or compounds, as well as with drugs and therapeutic agents already in use in the treatment of retinitis pigmentosa. The inventive method described here can be used on patients suspected or in the early stages of retinitis pigmentosa development with or without other oculopathies. This invention also envisions the use of this method as a prophylactic on patients in which the patients are predisposed to the retinitis pigmentosa condition.

### BACKGROUND OF THE INVENTION

One of the most devastating conditions affecting the retinal rods is “retinitis pigmentosa,” an inherited disorder which the rods gradually degenerate where the rods become dysfunctional affecting vision. The chief function of the retina is transduction (conversion) of light into nervous impulses by the rods and the cones. Retinitis pigmentosa is a chronic retinal degeneration where the deterioration is accompanied by abnormal deposits of pigment in the rods of the retina. The disease causes a progressive decrease in peripheral vision which this type of vision is the side vision. Eventually, the person with retinitis pigmentosa can see only straight ahead which the patient experiences a condition known as “tunnel vision”. The retinitis pigmentosa was recognized which the condition of RP (retinitis pigmentosa) was classified midway through the last century. There is little known about the causes of RP, the progression, and the treatment of RP.

Retinitis pigmentosa (RP) is a group of inherited diseases that damage the light-sensitive rods and the cones which make up the outer layers of the retina. Rods provide side (peripheral) and night vision. The rods are affected more than the cones. The cones are concentrated in macula called fovea centralis. The foveal centralis provides color and clear sharp central vision, also, called foveal vision which this vision is necessary in humans for reading, for watching television, for driving, and with activities where visual detail is required. The fovea centralis includes parafovea and perifovea of macular regions. Macula lutea is devoid of blood vessels where the macula lutea receives oxygen and nutrition from choroidal BV, across the Bruch's membrane, and retinal pigment epithelium (RPE).

The prevalence of retinitis pigmentosa (RP) in The United States is about 1 in 4000. The worldwide prevalence of RP is about 1 in 3000 where some estimate the prevalence is 1 in 5000. The carrier state is recognized to be approximately 1 in 100. The present invention described herein can be used with known carriers to prevent the development of retinitis pigmentosa. The highest reported incidence of occurrence for RP is among the Navajo Indians where there is 1 in 1878 and the lowest is in Switzerland (1 in 7000). A multicenter population study of retinitis pigmentosa population is 45 years or older. The study found that 52% had 20/40 or better vision in at least one eye, 25% had 20/200 or worse vision, and 0.5% had no light perception. RP is diagnosed in young adulthood where RP can afflict anywhere from infancy to the mid 30s to 50s. The X-linked RP is expressed only in the male. These X-linked varieties point out those men may be affected to some extent more than women.

Light Perception by Photoreceptors

The retina is the light (photon) sensitive portion of the eye which the retina contains the photoreceptors (cones and rods). These are the photosensitive cells of the eye for detecting the light that we see. They perform light perception by use of light sensitive pigments which the light sensitive pigments are basically made of protein called opsin and a chromophore called retinene which the variant is of vitamin A. The rods contain rhodopsin which the pigment is in the rods. The cones contain iodopsin and they have three distinct photo pigments. The rods and cones respond to light where they transmit signals through successive neurons which the neurons trigger a neural discharge in the output cells of the retina and the ganglion cells. The visual signals are carried by the optic nerve to the lateral geniculate bodies where the visual signal is passed on to the visual cortex of the occipital lobe, where it is registered as a visual stimulus.

The following sequence of events takes place in the photoreceptors when the photons strikes the photoreceptors outer segments:

1. In darkness, opsin is bound firmly to retinene as rhodopsin of the rods.

2. When light intensity is increased, retinene changes its shape. This is a structural change from cis- to trans-form.

3. Opsin can't hold retinene due to this change. Retinene is lost where the process is called bleaching.

4. Generator potential is produced as a result when the membrane of rod is depolarized.

5. Generator potentials add to generate an action potential resulting in a nerve impulse which the impulse is fired to the brain through the optic nerve.

6. Rhodopsin is reformed when retinene resumes the original shape using the ATP energy from the mitochondria where the ATP is formed whether there is light or no light. It is estimated that it takes about 30 minutes to regenerate the bleached pigment. The Rhodopsin is ready to be bleached to generate light perception, again. The brain in fact can detect one photon of light (the smallest packet of energy available) being absorbed by a photoreceptor.

Bleaching of iodopsin in cones is similar to rhodopsin in rods, but more light is needed to cause an action potential to be fired in cones—i.e. Threshold intensity for cones is higher than rods. Hence, the rods are mainly used for dim light vision where cones are used for bright light vision.

Vitamin A is important for vision, and the lack of vitamin A would result in night blindness. The excess intake of vitamin A can become toxic which the vitamin can harm our body. The lack of Vitamin A isn't the only cause of poor vision. B Vitamins are essential for normal functioning of the retina. It is possible the photoreceptors which the photoreceptors are genetically defective where the photoreceptors produce large amounts of reactive oxygen species (ROS) which the ROS can't be reduced in the retina due to untimely or reduced supply of the ATP from the mitochondria. It is likely, that the genetic defect in retinitis pigmentosa is in the mitochondrion which the mitochondrion doesn't supply enough ATP to reconstitute the photo pigments and to pump out ROS. The result is ROS accumulation induced damage. In one sense, retinitis pigmentosa can be classified can be called a mitochondrial related genetic disease where more research is needed to confirm. A significant part of our invention is that the insulin will restore the mitochondrial function by the insulin's metabolic effects of rebuilding all the intracellular organelle which includes the mitochondrion, the endoplasmic reticulum, the Golgi apparatus, the lysosomes, nuclear membrane and the nucleus.

Retinitis pigmentosa (RP) is not a single disease, but a collection of genetic eye conditions with symptoms of night blindness, which RP precedes tunnel vision for many years. This is due to progressive retinal dystrophy with rods reduction due to the apoptosis in which the RP can lead to blindness. Many people with RP will not become legally blind until, they are in their fifties where these individuals maintain a quantity of sight through their lives. Others go completely blind from RP where some cases result with blindness in early childhood. Development and progression of RP is different in each case. Retinitis pigmentosa is due to abnormalities of the photoreceptors (mostly rods and maybe some cones) and/or the retinal pigment epithelium (RPE) covering of the retina. RP leads to progressive visual loss. For the first time, the Retinitis pigmentosa afflicted experience defective darkness adaptation or nyctalopia (night blindness). The vision defect is followed by reduction of the peripheral visual field (contributing to the term known as tunnel vision), and the loss of central vision which tunnel vision occurs, later, in the course of the disease.

Signs of Retinitis Pigmentosa

Detailed Opthalmological Examination reveals the mottling of the retinal pigment epithelium with black bone-spicule pigmentation is a pathognomonic of retinitis pigmentosa. Ocular features include waxy pallor appearance of the optic nerve head, thinning of the retinal blood vessels, cellophane maculopathy, and cystic macular edema. Subsequent, posterior sub capsular cataract may occur. The condition can be associated with other oculopathies.

Diagnosis of Retinitis Pigmentosa

The diagnosing of retinitis pigmentosa relies on tests which the examination of the fundus of the eye, the visual field, electroretinogram, fluorangiography, and visus examination. The fundus of the eye examination aims to evaluate the condition of the retina and to evaluate for the presence of the characteristic pigment spots on the retinal surface. Examination of the visual field makes possible to evaluate the sensitivity of the various parts of the retina to light stimuli. It will be useful to have an objective documentation of the difficulty in visual perception experienced by the patient. The electroretinogram (ERG) consists of recording the electrical activity of the retina in response to particular light stimuli which ERG makes possible distinct valuations of the functionality of the two different types of photoreceptors (i.e. cone cells and rod cells).

The ERG is a must for diagnosing retinitis pigmentosa when the illness is in its initial stages. The resulting trace is almost either very flat or overall absent. The fluorangiography is performed by means of the intravenous injection of a fluorescent substance where there is photography of the retina at different times. The fluorescent substance in blood arrives at the retina where the substance colors the arteries, the capillaries and the veins; makes them visible at their functional state of their walls. The use of our invention insulin prior to the procedure which the fluorescent substance demarks the afflicted blood vessel. With Use of our invention insulin, the BV markers dyes reveal their physiological or their pathological state and thus helps in the diagnosis of the various retinal afflictions including retinitis pigmentosa.

Visus examination permits a valuation of visual acuity. This examination consists where the patient reads letters of different sizes at a distance of three meters. The form of inheritance of RP is determined by taking a family history. Nearly 35 different genes or loci are known to cause “nonsyndromic RP”. RP isn't the result of another disease or part of a wider syndrome as described below:

Retinitis Pigmentosa and the Genetic Origin

Studies have shown that the retinitis pigmentosa is caused by mutations in the rhodopsin gene, the peripherin gene, and possibly in other genes within the rod. Mutations in the peripherin gene may be the cause of another devastating retinal disorder namely “macular dystrophy.” RP can be inherited in an autosomal dominant, autosomal recessive or X-linked manner. X-linked RP can be recessive, affecting first and foremost males, or dominant, affecting equally males and females, although, males are usually more mildly affected. Retinitis pigmentosa (RP) is one of the most common forms of inherited retinal degeneration which RP is characterized by the progressive loss of photoreceptor cells that the loss may lead to blindness.

In 1989, a mutation of the gene for rhodopsin, a pigment, that the pigment plays an essential part in the visual transduction cascade enabling vision in low-light conditions, was identified. Since these discoveries, more than 100 mutations have been found in this gene, accounting for 15% of all types of retinal degeneration. Mutations in four pre-mRNA splicing factors are known to cause autosomal dominant retinitis pigmentosa. 150 mutations have been reported to date in the opsin gene associated with the retinitis pigmentosa. One of the main biochemical causes of RP in the case of rhodopsin mutations is protein misfolding. In addition, molecular chaperones have been involved in RP. Our invention will help to prevent the misfolding of the outer segment involved in RP.

Pathophysiology of Retinitis Pigmentosa

Though the retinitis pigmentosa has been known for more than a century, it's pathogenesis isn't understood and RPS' treatment is still elusive. All we know is that the RP is typically a rod-cone dystrophy of the retina in which the genetic defects cause cell death (apoptosis) concentrated in the rod photoreceptors where cell death is less in the cones. There are about 120 million rods and 7 million cones in each eye that the disease can affect. There is shortening of the rod outer segments. The process of shortening is followed by loss of the rods. Most of the loss takes place in the middle and peripheral part of the retina.

In many cases, the rods degenerate which the rods have a tendency to be worse in the inferior segment of the retina. This finding suggests a role of constant light exposure, where the relation to the production of oxidant reactive oxygen species (ROS) resulting in the death of the photoreceptors due to their effect. Gravitational pooling of the metabolites, ROS with reduced supply of nutrients may play a role. The large numbers of rods are found in the midperipheral retina. The cell loss in this area tends to lead to peripheral and night vision loss. Cone photoreceptor death occurs in a similar manner to rod apoptosis with the shortening of the outer segments resulting in cell loss. Due to paucity of these photoreceptors in this area, only a few cones are affected.

Retinitis Pigmentosa Clinical History

The initial symptom in RP is night blindness (Nyctalopia), which is a painless and progressive. Nyctalopia is considered a feature of the disease. Patients might struggle with tasks at night or in dark places. There is a problem walking in dim lit rooms (e.g, movie theaters), difficulties driving in low light, sundown, misty and cloudy conditions where the individual needs a prolonged period of time to adapt from light to dark. In the early on, the peripheral vision loss is often asymptomatic. Other characteristic symptoms are the reaction to excessively strong light (dazzlement—temporarily deprivation of the sight). A gradual narrowing of the visual field which the field displays itself in the form of difficulty in perceiving objects situated on either side or stumbling over steps or other low down impediment. Such patients may report running into furniture or door frames. The patients struggle with sports such as tennis, softball, football and basketball where peripheral vision is required. Many patients with RP relate to seeing flashes of light (photopsia). The patients describes seeing small, iridescent, blinking lights similar to the symptoms of an ophthalmic migraine aura. However, in contrast to the patient with an ophthalmic migraine, the photopsia may be continuous rather than periodic. The physician has to rule out phenothiazines/thioridazine toxicity to diagnose retinitis pigmentosa. The course of the illness is gradual and variable which the illness leads to vision disability. The direction and the course of the disease can be monitored with computer-averaged and narrow-band passed filtered responses. These studies show that the patients whom ages range from 6 to 49 lose an average 16% of remaining full-field ERG amplitude per year. Cones and rods appear to be functioning normally for their number with their amounts of remaining visual pigment.

Physical Findings in Retinitis Pigmentosa Upon Examination

The common findings are: Vision changes where the Snelling visual acuity can vary from 20/20 to light perception. This is usually preserved until late in the disease. Pupil reaction can be normal or lacks a defect. Surprisingly, nearly 50% of adult patients develop posterior sub capsular cataracts. This is a hint that there is oxidative damage due to generation of free radicals by the light in both these conditions. The retinal fundus is unaffected early in the disease. Findings on examination include: Bone spicules—Midperipheral retinal hyper pigmentation in a characteristic pattern; Optic nerve waxy pallor appearance; Atrophy of the retinal pigment epithelium in the mid periphery of the retina with retinal arteriolar attenuation; loss of the foveolar reflex or an abnormal vitreoretinal interface. The patient may develop cystoid macular edema coupled with fast and potentially reversible loss of vision. Retinitis pigmentosa, on occasions, shows Retinitis Punctata Albescens, a variant of RP, present with yellow deposits deep in the retina rather than the normal increased pigmentation of the peripheral retina.

Retinitis pigmentosa can be associated with rod-cone retinal degenerations present with central macular pigmentary changes (bull's eye maculopathy). Choroideremia—an X-linked recessive retinal degenerative disease which the disease leads to the degeneration of the choriocapillares, the retinal pigment epithelium and the photoreceptor of the eye. The gyrate atrophy, an autosomal recessive disease, causing progressive chorioretinal degeneration resulting in blindness which the blindness is caused by a deficiency of ornithine-aminotransferase (OAT). This OAT deficiency is described as “atypical retinitis pigmentosa” by Jacobsohn in 1888 which OAT has large characteristic scalloped appearance areas of peripheral retinal atrophy.

Differential Diagnosis of Retinitis Pigmentosa

A thorough physical examination is useful to rule out retinitis pigmentosa which RP can be associated with pigmentary retinopathy that the pigmentary retinopathy mimics retinitis pigmentosa which this can be found in Usher Syndrome, Waardenburg Syndrome, Alport Syndrome, and Refsum disease; Kearns-Sayre Syndrome; Abetalipoproteinemia; mucopolysaccharidoses (e.g., Hurler Syndrome, Scheie Syndrome, Sanfilippo Syndrome); Bardet-Biedl Syndrome; Neuronal ceroid lipofuscinosis and others. These disorders have been categorized clinically in relation to the age of onset which the chronological relation of vision loss to neurologic symptoms.

Present Treatment Available for Retinitis Pigmentosa

There isn't a specific cure for the RP condition. The progressive evolution of the retinitis pigmentosa can be reduced by the daily intake of 15000 IU (equivalent to 4.5 mg) of vitamin A palmitate (Berson E L, Rosner B, Sandberg M A, et al. (1993). “A randomized trial of vitamin A and vitamin E supplementation for retinitis pigmentosa”. Arch. Opthalmol. 111 (6): 761-72). Recent studies have shown that appropriate vitamin A supplementation can postpone blindness by almost 10 years (Berson E L (2007). “Long-term visual prognosis in patients with retinitis pigmentosa” the Ludwig von Sallmann lecture Exp. Eye Res. 85 (1): 7-14). Scientists continue to investigate possible treatments without much success. Future treatments may involve retinal transplants, artificial retinal implants, gene therapy, stem cells, other nutritional supplements, and/or drug therapies. Our invention brings therapy that the therapy will hold back the photoreceptors' degeneration and the progression of the disease and curtail or cure the condition.

Scientists at the Osaka Bioscience Institute have identified a protein, named Pikachurin, which they believe could lead to a treatment for retinitis pigmentosa (Sato S, Omori Y, Katoh K, et al. (August 2008). “Pikachurin, a dystroglycan ligand, is essential for photoreceptor ribbon synapse formation”. Nat. Neurosci. 11 (8): 923-931). Attempts have been made at University College London Institutes of Opthalmology to treat retinitis pigmentosa with stem cell implant in mice resulting in photoreceptors development with the necessary neural connections. Previously, there was belief that the mature retina has no regenerative ability. These findings aren't available to treat retinitis pigmentosa in humans yet.

It is important to note that the diseases that affect photoreceptors do not harm retinal ganglion cells (which convey the visual impulses from photoreceptors to occipital cortex through lateral geniculate body), and vice-versa. Biochemical pathways are active in one cell population where this doesn't take place in the other and vice-versa. The two layers of retina receive completely independent vascular supplies. The ganglion cells are nourished by the retinal blood vessels which the blood vessels originate from the central retinal artery where the artery penetrates the eye through the optic nerve disc which the artery branches off into smaller vessels that covers the innermost layers of the retina where they are embedded in the ganglion cell and the optic nerve fiber layer. These vessels don't play a role in the support of photoreceptors which the photoreceptors are nourished by the underlying choroid by controlled diffusion from choriocapillares that is regulated by the Bruch's membrane and the retinal pigment epithelium (RPE).

The pigment epithelium and the Muller cells are the most important radial glial cells of the retina which the glial cells are essential for photoreceptor functioning and homeostasis. The glial cells ensheath the photoreceptors from the synaptic terminus to the level of the photoreceptor inner segment where the glial cells form a very close physical relationship. The relationship is akin to oligodendroglia and the Schwann cells which enclose the axons in the central and the peripheral nervous system. This has been demonstrated that Muller cells offer metabolic and trophic support to photoreceptors which the Muller cells support their continued existence and survival. This is especially true for rod photoreceptor cells which they originate from the same precursor as Muller cells. These cells support photoreceptors by buffering and by protecting the local microenvironment from surplus extracellular potassium and glutamate, may be even ROS that accumulates as a result of the photo transduction cascade. The neurotransmitter releases these at the synaptic station respectively. The glutamate released at the photoreceptor synapse is internalized by the Muller cells by means of high-affinity carrier systems which the glutamate is converted to the non toxic amino acid glutamine by glutamine synthetase. This enzyme is widely represented throughout the whole group of the Muller cells. Our invention of using insulin ophthalmic instillation can help to restore the glutamine synthetase regenerative ability in these overloaded Muller cells and maintain the integrity of the rods preventing and/or delaying their apoptosis as seen in retinitis pigmentosa due to excessive accumulation of glutamate. Glutamate causes excitotoxicity in the CNS; and the retina being an extension of the brain, the glutamate produces the same excitotoxicity of the retina.

It was discovered that bendazac, or (1-phenylmethyl-1IH-indazol-3-yl) oxy acetic acid, when this was administered in a form suitable to obtain therapeutic tissue concentrations which results determined an improvement in retinitis pigmentosa. The lysine salt of bendazac was used due with good oral absorption; Dosage was 500 mg three times a day. They used up to 1% solution as ophthalmic drops.

### SUMMARY OF THE INVENTION

The invention relates to a method of instilling insulin ophthalmic drops in the conjunctival sac for treating retinitis pigmentosa due to any etiological factors both genetic and non genetic.

The retinitis pigmentosa is treated with Insulin and/or IGF-I with or without known anti-retinitis pigmentosa therapeutic, pharmaceutical, biochemical, and biological agents or compounds.

The invention furthermore uses this method as prophylactic on patients where the patients are predisposed to develop retinitis pigmentosa.

The invention additionally treats other oculopathies associated with and/or contributing to retinitis pigmentosa.

The present invention describes the retinitis pigmentosa development, signs, symptoms, pathophysioligy and treatments available.

One aspect of the present invention is a method for treating the retinitis pigmentosa in humans and mammals by administering to the afflicted eye. The therapeutic effective dose of insulin instilled into the conjunctiaval sac.

The invention encompasses synergistic combinations which the opthalmic preparation has the insulin that the therapeutic efficacy is greater than an additive. If possible, the mixture reduces or avoids unwanted or adverse effects.

In certain embodiments, the combination therapies encompassed by the invention provide an improved overall therapy relative to administration of an active insulin compound or any therapeutic agents or treatment alone. In certain embodiments, doses of existing or experimental ophthalmic therapeutic agents or treatments can be reduced or administered less frequently where the invention increases patient compliance which the compliance improves the therapy and reduces the unwanted or adverse effects.

Other features and advantages of the instant invention will become apparent from the following description of the invention which refers to the accompanying drawings.

### BRIEF DESCRIPTION OF THE DRAWINGS

FIG. 1 is the diagram of the longitudinal section of the eye 200 showing conjunctival sac 202 containing the insulin drops.

FIG. 2 is the drawing of the longitudinal section of the eye 300 showing the structures involved in the production and drainage of aqueous humor which transports insulin and other therapeutic agents.

FIG. 3 is the diagramatic presentation section of the anterior part of the eye 500 presentating the rich vascular plexus which transports the insulin.

FIG. 4 is the diagramatic presentation 400 showing the vascular arrangement of the choroid surrounding the retina.

FIG. 5 is the diagramatic presentation 700 showing the histology of the retina and its blood supply.

FIG. 6 is the diagramatic presentation 600 showing the conjunctival formix and the route of drainage of therapeutic agents to the nose.

FIG. 7 is the diagrammatic presentation 800 showing the histology of the retina, its relation to the blood supply, and the route of Insulin and IGF-1 transfer to photoreceptors.

### DETAILED DESCRIPTION OF THE INVENTION

The ophthalmic drops or preparations to be used to treat retinitis pigmentosa should be stable, dissolved or solubilized which the preparation is safe and effective with opthalmological standards in place. The term ‘stable’, means physical, rather than chemical stability with no crystallization and/or precipitation in the compositions, when the preparation is stored at a refrigerated or room temperature. The preparation comes in contact with lacrimal secretions when the preparation is applied to the conjunctival sac and the cornea. The label ‘dissolved’, ‘dissolving’, ‘solubilized’ or ‘solubilizing’, means that an ingredient is substantially solubilized in the aqueous composition without the particulate, crystalline, or droplet form in the composition.

The phrase ‘opthalmological acceptable’, refers to those therapeutic, pharmaceutical, biochemical and biological agents or compounds, materials, compositions, and/or dosage forms suitable for use in a mammalian eye without undue toxicity, irritation, allergic response, or other problem or complication, commensurate with a reasonable benefit/risk ratio. The expression ‘safe and effective’, as used herein, means a concentration and composition, that the concentration and composition is sufficient to treat without serious local or systemic side effects. The term “ocupopathies” means any and all diseases affecting the eye lids, eye ball with retina, optic nerve, choroid, eye ball, and their function.

The following diagrams describe the structure of the eye, and explain the route of movement, transportation, and diffusion of insulin where other therapeutic agents instilled in the conjunctival sac topically for the treatment of retinitis pigmentosa.

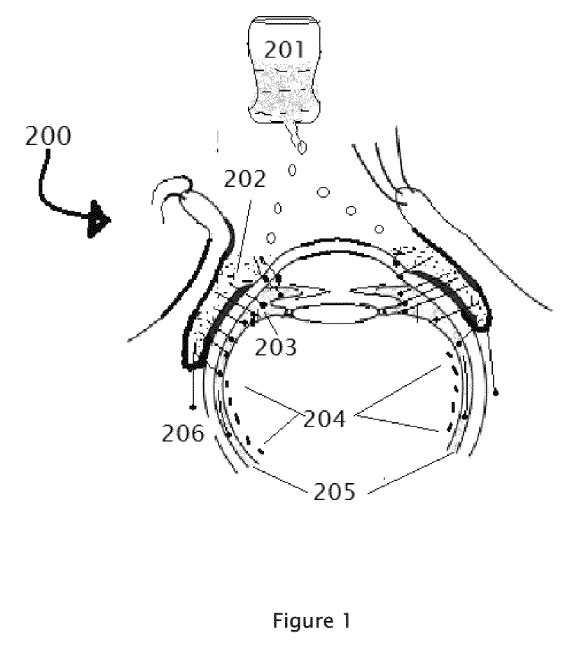

FIG. 1 is the diagram of the longitudinal section of the eye 200 showing conjunctival sac 202 containing the instilled insulin drops. The other therapeutic agents introduced through a dropper 201 and their passage to iridocorneal angle, anterior and posterior chambers, iris, ciliary body, and processes 203, choroid, and the anterior segment of the retina 204 which the other therapeutic agents contains photoreceptors rods. The photoreceptors are affected by the retinitis pigmentosa (drumstick markers). Note: the ophthalmic insulin eye drops and other therapeutic agents pass on to the choroid 205 adjacent to the retinal pigment epithelium and retinal photoreceptors. This delivers the therapeutic agents to the afflicted rods. The therapeutic agents' passes through the episcleral plexus of veins to the periphery of the sclera 206 where the agents can be reabsorbed which the agents circulate back into the choroid and retinal blood vessels (BV).

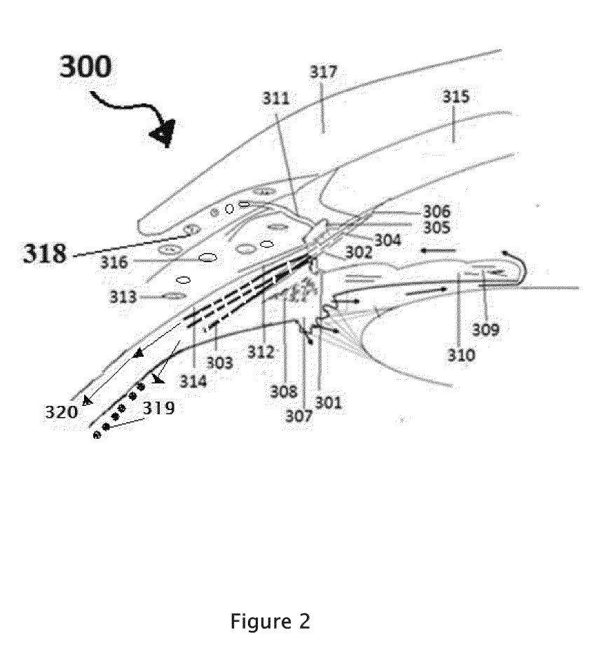

FIG. 2 is the drawing of the longitudinal section of the eye 300 showing the structures involved in the production and the drainage of aqueous humor which the structures pick up the therapeutic agents including insulin used in the treatment of retinitis pigmentosa. The insulin circulates through various sites of action where the therapeutic agents reach their ultimate site of action with ease to the retinal rods (arrows). The therapeutic agents entering the anterior chamber aqueous humor through the subconjunctival, 313,316,318, episcleral arteriovenous plexus 313,316 pass through the uveoscleral meshwork 301, Corneoscleral meshwork 302, Juxtacanalicular or cribriform trabecular meshwork 304, Schlemm's canal 305, Corneal endothelium joining the trabecular meshwork 306, Longitudinal 303, and circular fibers of the ciliary muscles 308; muscle fibers of the iris 309, 310, Scleral sinus vein 311, Scleral Spur 312, Scleral Veins 313,316, Suprachoroidal space between choroid and sclera 314. The cornea 315 and sclera 316 participate where the least of the therapeutic agent's circulation or transportation, except, at the cornea-scleral junction. The conjunctival sac 317 (formix) where the insulin is with the therapeutic, pharmaceutical, biochemical, and biological agents, or compounds are deposited to be transported (arrows) to the retina through the ciliary body 307, trabecular mesh work, choroid, and irido-scleral angle 301, choroid plexus projecting from the ciliary body 307. The choroid plays an important role in transporting the insulin and therapeutic agents (arrows) to the retinitis pigmentosa afflicted retina 319 (From Shantha T R and Bourne G H. Some observations on the corneal endothelium. Acta Opthalmologica 41: 683-688: 1963).

This diagram illustrates how easy it is for the insulin and other selected therapeutic agents to reach the afflicted retinitis pigmentosa site 319 from the conjunctival sac (arrows). From conjunctival sac 317, the therapeutic agents enter into the anterior chamber, corneal endothelium 306, 304, trabecular meshwork 301, 302, and ciliary body 308 passing through the sub and inter conjunctival blood vessel plexus of the eye 313, 316, 318, choroid 320, suprachoroidal space 314 where they reach their destination 319 to have therapeutic effect on the retina involved in retinitis pigmentosa. The arrows markers indicate the site of entry and the circulation of the insulin and other therapeutic agents from the conjunctival sac where they exert their effect in the treatment of retinitis pigmentosa.

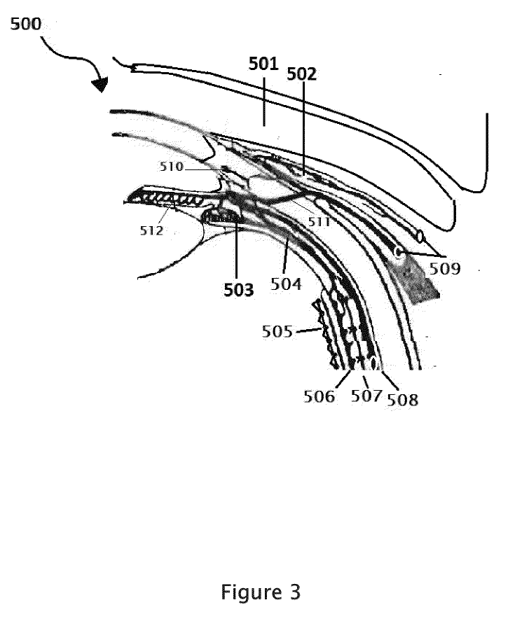

FIG. 3 is a diagramatic section of the anterior part of the eye 500 presentating the rich vascular plexus which the plexus is responsible for transporting the insulin and other therapeutic agents from the conjunctival sac 501 to the site of retinitis pigmentosa 505. Note: the rich vascular plexus 502 under the conjunctiva of the eye ball which the vascular plexus helps to transport the therapeutic agents from the conjunctival sac 501 to intrascleral 511 veins and canal of Schlemm 510 with the venous connection, various vascular structures of iris 512, iridocorneal angle, ciliary body with the ciliary processes 503 where there is a rich BV, and finally passes to the choroid vascular plexus 504, 507, retinal pigment epithelium 506, supra and inter choroidal space 508 where the therapeutic agents reach the base of the rods of the retina 505, the site of the retinitis pigmentosa. Note: the rich vascular plexus of the iris 512, choroid, ciliary body 503, which these communicates with the subconjunctival BV 502, suprachoroidal space 508, and choroidal vascular net work 504,507 where the choroidal vascular network delivers insulin and anti retinitis pigmentosa therapeutic agents to various structures between the ciliary body and the iridoslceral angle and scleral-corneal space, and supra scleral network of vascular plexus 509 finally reaching the retina. This diagram shows the vascular net work under the conjunctival sac which this delivers the insulin. The therapeutic agents are delivered to the site of retinitis pigmentosa 505 through various vascular plexus to the afflicted rods which the defective so as to cause the visual defects, disability and blindness.

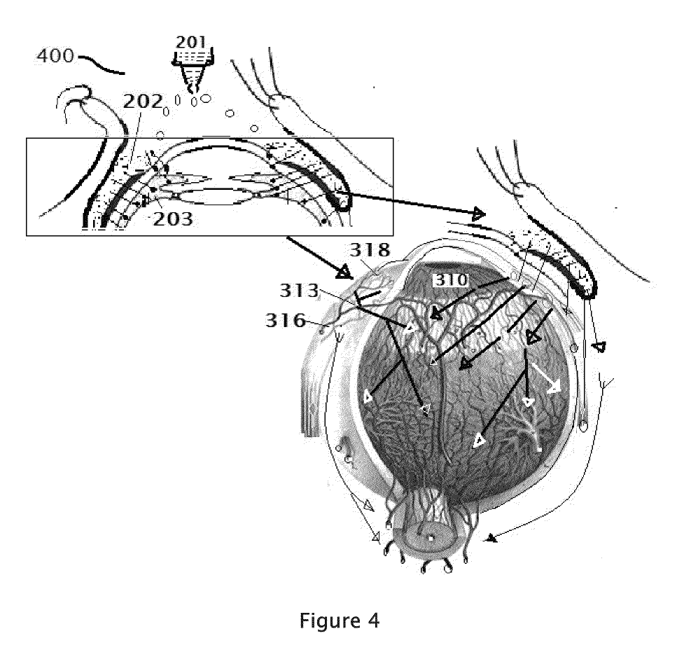

FIG. 4 is the diagramatic presentation 400 showing the vascular arrangement of the uveal track which the uveal track is the middle layer of the eye divided into from front to back, as the iris 310, ciliary body 203, and the choroid (arrows) that covers the entire retina in which these structures are involved in the transfer of insulin and other therapeutic agents to the retina—to the sites of the RP. These three structures of the uveal system are highly vascular where these structures communicate with the subconjunctival 318 and scleral vessels 313,316. The entire uvea is soaked in aqueous humor as it permeates from the anterior chamber to the choroid through the trabecular meshwork. The insulin and the therapeutic agents 201 from the conjunctival sac 202 are transported to the sub conjunctival venous plexus 318 inter and epi scleral veins 313,316 and retro bulbar veins surrounding the optic nerve as it exits the eye (long arrows at the optic nerve). The therapeutic agents are transported to the uveal vascular plexus (multiple drumstick and plain arrows). This rich vascular plexus transports the therapeutic agents to the retina through the retinal pigment epitheliums. The blood vessels of the uvea are involved in the health of the retina by transporting and by providing proper nurticeuticals; at the same time, the metabolites are removed for the photoreceptors. In the same fashion, they carry insulin and the other therapeutic agents which they deliver to the retinal rods, the site of retinitis pigmentosa. This diagram shows, how efficiently, the insulin and the other therapeutic agents from the conjunctival sac 202 are absorbed and transported to the subconjunctival, scleral vascular plexus 318, 313,316; delivered to the uveal system (arrows) including iris 310, and then to the retina, the site of retinitis pigmentosa pathology. Arrows points to the spread of therapeutic agents from the conjunctival sac to the rich uveal vascular network. Arrows shows that some of the therapeutic agents are transported to the supra scleral space where the therapeutic agents are transported back through the penetrating arterio-venous net work on the optic nerve (arrows) and posterior surface of the sclera (Based on Grays Anatomy diagram 7.255 on the histology of the eye).

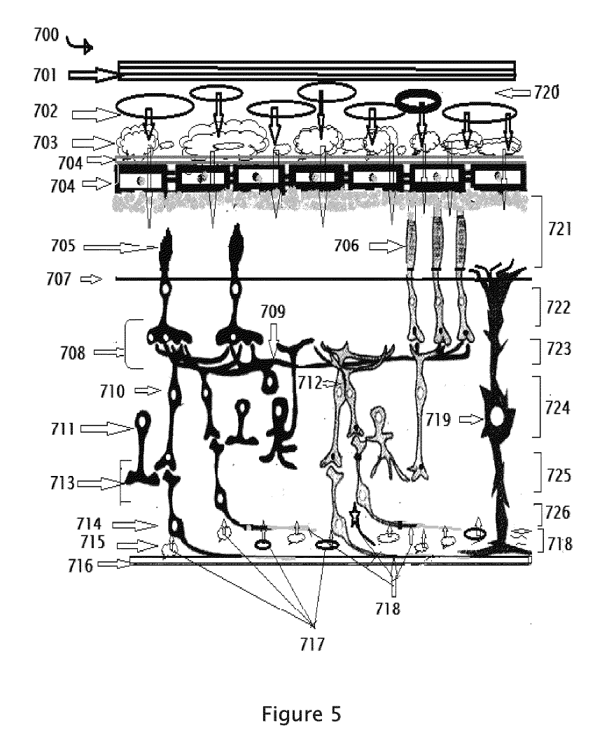

FIG. 5 is the diagramatic presentation 700 showing the histology of the retina in relation to the blood supply and trasfer of therapeutic agents to the site of RP. Our invention of the use of insulin and other therapeutic agents reach the rod and cone photoreceptors cells involved in the disease of retinitis pigmentosa. It shows sclera 701, large choroidal blood vessels 702, fenestrated choriocapilareis 703 which the choroidal blood vessels delivers the insulin and the other therapeutic agents (indicated by multiple large and the small arrows directed downwards towards rods and cones) which the choroidal blood vessels carry oxygen and nutriceticals, through the noncelluar Bruch's membrane 704, which the noncellular Bruch's membrane acts as a interface between the pigment epithelim 704 and choriocappillaries 703. This separates retinal pigment epithelium form the choriocapilaries 703. The cones 705 are not in intimate contact with the retinal pigment epithelium 704. The rods are in close contact with the retinal pigment epthelium brush border 704. The outer limiting membrane 707 is formed by the Muller cells 719. It separates the photoreceptors outer segments from the rest of the retina which the separation may prevent the transfer of components from extracelluar space of the photoreceptors to the rest of the retina.

In the same fashion, the therapeutic agents get concentrated as they are transported from choriocapillaries towards the outer segment of the photoreceptors which this is the site of the retinitis pigmentosa pathology. Note: the outer plexiform layer 708, and horizontal cells 709 are the laterally interconnecting neurons in the outer plexiform layer of the retina, which the above structures modify and integrate the signals from the rods and cones where the rods and the cones are responsible for allowing eyes to adjust where the patient can see equally in bright and dim light conditions. They help to integrate and regulate the input from multiple photoreceptor cells. The bipolar cells 710,712 are situated between photoreceptors (rods 706 and cones 705) and ganglion cells 714. The therapeutic agents from the conjunctiva do not reach these cells in high concentration due to the presence of outer limiting membrane and paucity of vascular network. They act, directly or indirectly, to transmit signals from the photoreceptors to the ganglion cells.

Amacrine cells 711 are the interneurons (40 types are recognized) are responsible for 70% of the input to retinal ganglion cell 714. The bipolar cells 710, 712, are responsible for the other 30% of input to the retinal ganglion cells. The inner plexiform layer 713, ganglion cell layer 714 receives the signals from the rods and cones. The inner retinal blood vessels 717 supply oxygen and nutrients to the inner part of retina. The inner retinal blood vessels are shown by mulitiple short arrows pointed towards outer side of the retina. The optic nerve fibers 718 derived from the gangion cells 714 relay the photoreceptors signals to the CNS. Note: the Muller cell 719 extends to contributes to the inner limiting membrane 716 separating the vitreous from the retina and the outer limiting membrane 707.

The arrows from choroid indicate the rich vascular supply to the outer segments of the photoreceptors (compared to the rest of the retina), which the outer segments receive the therapeutic agents from the conjunctiva compared to the paucity of BV from the retinal inner BV 717. This diagram shows the insulin and other therapeutic, pharmaceutical, biochemical and biological agents or compounds from conjunctiva and chorid blood vessels have easy access to rods 706 and cones 705 outer segments in the treatment of retinitis pigmentosa. The insulin and other therapeutic agents of our invention are transported by the aqueous humor through the suprachoroidal space where the agents permeate to the space between the retinal pigment epithelium and the photoreceptors. The inner limiting membrane 716 is the boundary between the retina and the vitreous body which the inner limiting membrane is formed by astrocytes and the end feet of Muller cells 719. The membrane is separated from the vitreous humor by a basal lamina.

There may be some leaking of aqueous humor from ciliary epithelium and zonule fibers containing insulin and other therapeutic agents seeps between these two structures through this basal lamina. The transport or soaking has to be minimal. If it does seep, the concentration is mostly at mid and anterior part of the lower segment (between 5-7^{O}-clock positions) of the retina due to gravitational drag where the pathology of retinitis pigmentosa is pronounced besides the mid and anterior part of the retina which are the main parts affected by the RP.

This diagrams 700 shows various histological layers of the retina which are as follows: layer of retinal pigment epithelium 704, layer of rods and cones 721, outer nuclear layer 722 made up of nuclei from rods and cones, outer limiting membrane 707 formed by Muller cells, outer plexiform layer 723 made up of synapses between the rods and cones with horizontal and bipolar cells. The inner nuclear layer 724 made up of bipolar and amacrine cell nuclei, inner plexiform layer 725 formed by synapses between the ganglion cells 714, 726 and the process of cells from the inner nuclear layer. The nerve fiber layer formed by the axons of the ganglion cells grouped to become the optic nerve where the nerve fiber leaves the eye at the optic disc to lateral geniculate bodies then to the occipital cortex.

The inner limiting membrane 716 is made up of Muller cells expanded inner feet and astroglia. The diagram shows how each retinal layer is in touch with the blood vessels. Their supply of nurticeuticals, oxygen, insulin, and other therapeutic agents used in the treatment of retinitis pigmentosa. It is clear that the outer segment of the photoreceptors get the most exposure to the therapeutic agents compared to other functional units of the retina.

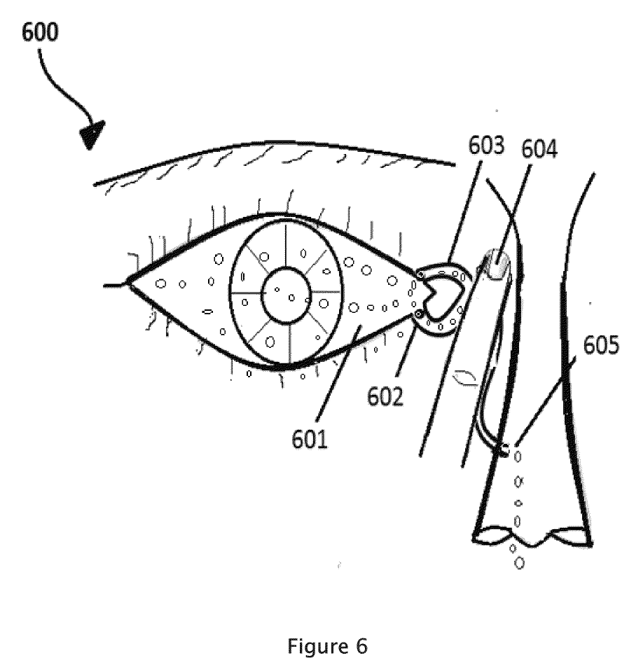

FIG. 6 is the diagramatic presentation 600 showing the route of drainage of the lacrimal fluid and therapeutic agents shown as bubbles from the conjunctival formix (sac) 601 to the nasal mucosa 605 and illustrates a method to prevent the agents from entering the nasal mucosa. A simple method applying the finger pressure 604 at the medial eye angle and nasal junction. The location of the lacrimal punctum, canaliculi 602, 603 and lacrimal sac with a finger 604 will prevent the therapeutic agents drainage to the nasal cavity and the nasal mucosal absorption 605, and their associated systemic adverse effects.

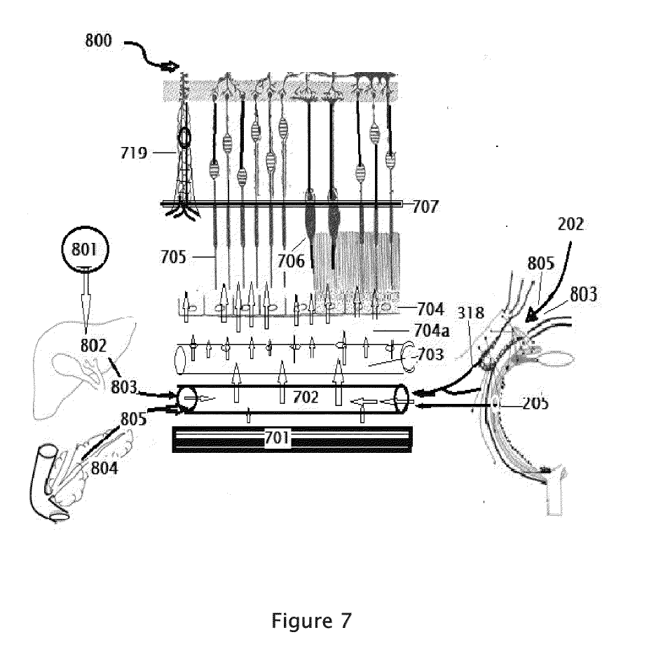

FIG. 7 is the diagrammatic presentation 800 showing the histology of the external layers of the retina including photoreceptors and their relation to the blood supply. Our invention of Insulin and IGF-1 and other therapeutic agents can reach from the systemic blood supply and conjunctival sac of the eyes to reach the rods and cones photoreceptors cells involved in the pathogenesis of the disease retinitis pigmentosa. It shows sclera 701, large choroidal blood vessels 702, fenestrated choriocapillareis 703 which the capillaries deliver the therapeutic agents insulin 805 and/or insulin like growth factors 803 from the ophthalmic drops 202. Liver 802 makes IGFs from growth hormone 801 from the pituitary gland. The pancreas 804 secrets insulin hormone 805 which the insulin hormone enters the circulation through the portal circulation. Then the insulin and IFG-1 reach the retina through the blood supply to the eye ball and retina.

The ophthalmic drops 202 of insulin 805 and IGFs 803 are absorbed by the subconjunctival blood vessels 318 and choroid 205. The growth hormone from the pituitary gland is converted to IGFs where the IGFs are circulated to reach all over the body including choroidal BV 702, choriocapillares 703, ultimately, reaches the retina. The insulin 805 is produced by the pancreas 804 where it reaches the choroidal BV 205 where it, also, reaches the retinal photoreceptors 705, 706. The Insulin and IGF-1 from the choroidal BV 702 pass to the fenestrated choriocapillares 703 which the choriocapillaries are leaky. The leaked fluid from the inside to extracellular space of the Bruch's membrane 707a, passes through the retinal pigment epithelium (RPE) 704 to reach the outer segments of the photoreceptors 705, 706. The extracellular fluid is bound by RPE and the external limiting membrane 707 formed by the Muller cells 717. The big and small arrows show the directions of the flow of Insulin and IGF-1 from the conjunctival sac 202 where there is the systemic circulation from liver and pancreas.

The arrows from the choroid indicate the rich vascular supply to the outer segments of the photoreceptors which the photoreceptors receive the therapeutic agents from the conjunctiva compared to the paucity of BV from the retinal inner BV. This diagram shows that the therapeutic, pharmaceutical, biochemical, and biological agents or compounds from conjunctiva and chorid blood vessels have easy access to rods 706 and cones 705 in the treatment of retinitis pigmentosa. The therapeutic agents are transported by the aqueous humor through the suprachoroidal space where the agents permeate to the space between the retinal pigment epithelium and the photoreceptors.

Any treatment of retinitis pigmentosa with or without other oculopathies with ophthalmic topical preparations (eye drops) designed in our invention (as well in other inventions) using Insulin and/or IGF-1 and other therapeutic agents as prophylactic, and/or for treatment encompasses the following principles: 1. Eye drops, semi liquids, gels or ointments should act like a film covering like natural tears over the ocular surface of the eye including cornea with less stinging or burning sensation, 2. The above are capable of providing mechanical lubrication for the ocular surface which the eye lid glides easily during the blinking movement. 3. The reduction of the evaporating natural lacrimal fluid, 4. The emulsion or the watery ophthalmic drops shouldn't react with eye cellular structures, the lacrimal coating, and the eye lid lacrimal glandular system, 5. Eye drops should be stable for a reasonable period of time at room temperature. 6. The therapeutic preparations should be easily absorbed with or without other absorption enhancers and transported to the site of the pathology. 7. Besides acting against retinitis pigmentosa pathology, the therapeutic preparations should contain therapeutic, pharmaceutical, biochemical and biological agents or compounds capable of alleviating the underlying cause responsible for RP; at the same time augment/amplify the effects of therapeutic agents with trophic effects when used with our invention. In our invention, insulin is based on meeting all the above recited physiological, pharmacological, and therapeutic parameters.

Before, the explanation and the description of the disclosed embodiments of the present invention in detail, which it is to be understood that the invention is not limited in its application to the details of the particular examples and arrangements shown. Since the invention is capable of other examples and embodiments in treating other oculopathies. The terminology used, herein, is for the purpose of description and not to the limitation. As earlier enumerated above and narrated below: this application has been filed in order to disclose: Insulin and Insulin-like Growth factor (IGF-1) have been found to have high therapeutic activity against metabolism of the cells. All its functions including retina and photoreceptors involved in retinitis pigmentosa. Insulin and/or IGF-I not only restores the proper physiological functioning of the retina by acting against the etiological factors such as ROS, genetic defects, correcting any mitochondrial metabolic defect, and restoring the membrane stability; it enhances the effectiveness (augmentation-amplification effects) of other therapeutic, pharmaceutical, biochemical, and biological agents or compounds already used in the treatment of retinitis pigmentosa and other oculopathies.

Insulin, as applied in the present invention, helps to maintain functional and structural integrity of the photoreceptors even though they have genetic defects. Furthermore, our invention insulin helps to delay the expression of genetic defects that these genetic defects exist in the photoreceptors which these genetic defects predisposes or causes the retinitis pigmentosa.

At present, insulin is exclusively used to treat type I and certain cases of type II diabetes. Our discoveries and inventions describes the use topically (locally) in other disease conditions other than diabetes including cancers, dry eye syndrome, glaucoma, prostate diseases, middle and inner ear afflictions, CNS diseases including Alzheimer's, to treat hair loss, enhancing eye lashes, activating vaccines, cytokines, Lymphokine, monoclonal antibodies; activating local immune system at lymph nodes, enhancing the local effects of chemotherapeutic agents, in treatment of autoimmune diseases, age related changes of the facial skin, healing of wounds, gum diseases, local infections and multiple local and systemic therapeutic applications.

Insulin and its Biological Effects on Healthy and Disease Afflicted Cells, Photoreceptors Cells in Retinitis Pigmentosa.

The Role Insulin Plays with the Uptake, Distribution; Augmentation-Amplification Effects of Therapeutic, Pharmaceutical, Biochemical and Biological Agents or Compounds On Photoreceptor Cells are Described Herein.

A variety of carriers, adjuvant agents, absorption enhancers, and facilitators, assist to get entry into the cell, potentiators of therapeutic action (augmentation/amplification effects), cell metabolic activity enhancers, cell multiplication enhancers, and other methods have been used to enhance the absorption and/or to potentiate the effect of therapeutic, pharmaceutical, biochemical, and biological agents or compounds administered to the patients for improving the physiological function, and the treatment of diseases. Discovery of insulin described in this invention is such a biological agent which we give details and elaborate below.

In 1921, the medical researchers, Drs. Frederick Banting and Charles Best at University of Toronto physiology department; isolated insulin from dog pancreas and tested this on diabetic dogs, successfully lowering the dogs' blood sugar level. On Jan. 11, 1922, Leonard Thompson, a 14-year-old boy who was dying of diabetes, was given the first human experimental dose of insulin. He lived 13 more years and died at the age of 27 from pneumonia, not from diabetes.

Besides Aspirin and antibiotics, insulin is the most commonly used therapeutic agent known to the public and professional alike. Insulin is a hormone secreted by beta cells of the islets of Langerhans in the pancreas. It has been self administered in home by the patient or in the office by the physician to treat diabetes. Insulin can be easily obtained by prescription which the insulin can be used for treating retinitis pigmentosa as described in this invention. So far, there are no reports of using the insulin as therapeutic agent locally to treat localized diseases or parentarily to treat systemic diseases other than diabetes. The present inventor is the first person to experiment with the use of insulin locally for almost a decade to treat many kinds of diseases of various tissues and organs in the body including cancers, and diseases of the ear, eyes, prostate, teeth, gums, CNS, eyes, hair growth, and other such conditions with many known therapeutic, pharmaceutical, biochemical, and biological agents or compounds.

In 1965 Sodi-Pollares et al. for the first time used glucose-insulin-potassium (GIK) solutions to treat patients with acute myocardial infarction. He found that GIK limited infarct size, reduced ventricular ectopy, and improved survival (Sodi-Pollares D, Testelli M D, Fisleder B L. Effects of an intravenous infusion of a potassium-glucose-insulin solution on the electrocardiographic signs of myocardial infarction. Am J Cardiol. 1965; 5:166-81). Insulin benefits the post ischemic myocardium by stimulating pyruvate dehydrogenase activity, which this activity in turn stimulates aerobic metabolism on cardiac and other tissue reperfusion. Exogenous insulin helps to reverse insulin resistance during cardiopulmonary bypass, which the exogenous insulin contributes to increased serum concentrations of free fatty acids and decreased myocardial uptake of glucose and increased myocardial function.

Intravenous direct infusions of insulin after coronary artery bypass graft surgery (CABG) have been shown to decrease the levels of free fatty acids and increase myocardial uptake of glucose. Insulin added to antegrade and retrograde tepid (29° C.) blood cardioplegia during coronary artery surgery has been shown to stimulate aerobic metabolism during reperfusion, preventing lactate release and improving left ventricular stroke work index with the restarting of the heart beating without many arrhythmias. This is the report of using insulin locally on a dynamic large organ, the heart. You can imagine the effect of insulin at cellular level of small structures such as eye, when insulin has profound effect on a massive dynamic organ like the heart! Insulin is especially beneficial for patients with diabetes and acute coronary ischemia (Svensson S, Svedjeholm R, Ekroth R. Trauma metabolism of the heart: uptake of substrates and effects of insulin early after cardiac operations. J Thorac Cardiovasc Surg. 1990; 99:1063-73. Rao V, Mississauga C N, Merrante F. Insulin cardioplegia for coronary bypass surgery [abstract]. Circulation. 1998; 98 (Suppl):I-612). Insulin increases the glutathione synthesis by activating gamma-glutamyl-cysteine synthetase. The insulin metabolic effects which the insulin reduces both polymorphonuclear neutrophils adhesion due to ROS (reactive oxygen species) can be effective in post perfusion adhesion of white blood cells to ROS with resultant cellular damage and stimulated tyrosine phosphorylation.

Reactive oxygen species (ROS) are reactive reckless molecules that contain the oxygen atom to include oxygen ions and peroxides which they can be inorganic or organic. They are highly reactive due to the presence of unpaired valence shell electrons where the electrons produce hydrogen peroxides which cause cell damage due to the cell membranes by peroxidation. Photoreceptors and other cells are able to defend themselves against ROS damage through the use of superoxide dismutase's, catalases, lactoperoxidases, glutathione peroxidases, and peroxiredoxins. Small molecule antioxidants such as ascorbic acid (vitamin C), tocopherol (vitamin E), uric acid, polyphenol antioxidants, and glutathione. These play important roles as cellular antioxidants to protect against ROS. The most important plasma antioxidant in humans is uric acid. H₂O₂ induced lipid peroxidation was greatly inhibited by insulin pretreatment.

Insulin increased redox status by increasing intracellular glutathione (GSH) content in oxidized cells. This reduced the ROS from the cells. The results show that GSH can reverse the effect of oxidation (oxidative free radical damage) on tyrosine kinase activation and phosphorylation. Thus, GSH plays an important role in cell signaling, which confirms the antioxidant activity of insulin to prevent the photoreceptors damage by ROS. This is a signal that insulin plays an overwhelming role in maintaining homeostasis. Insulin improves cellular physiological function in addition that the insulin augments/amplifies the effects of therapeutic agents when the insulin is used locally as described below in this invention at localized tissue levels, in the cornea, retina, and in the eye ball. Hence, our invention, with local use of insulin alone or with other therapeutic agents, is very effective in treating RP and related afflictions of the retina.

Insulin affects the DNA, RNA, and protein synthesis which results in increased growth by mitosis (Osborne C K, et al. Hormone responsive human breast cancer in long-term tissue culture: effect of insulin. Proc Natl Acad Sci USA. 1976; 73: 4536-4540); enhances the permeability of cell membranes to many therapeutic agents besides glucose, and electrolytes; Insulin helps and facilitates to move the drugs and therapeutic agent molecules from extra cellular fluid (ECF) to intracellular fluid (ICE) meaning from outside the cells to inside the cells which this facilitation can be seen in the use in coronary artery bypass graft (CABG) surgery as described above. In our studies of the local effects of insulin, the fact is that the growth hormone is ineffective in the absence of insulin. That is why the insulin with or without growth hormone is one of the most important biological agents to maintain the health and the functions of all the cells including photoreceptors which the photoreceptors are affected in retinitis pigmentosa.

Insulin and IGFs have properties of tissue growth factors, but, they have additional well recognized functions as hormones where the hormones regulate growth and energy metabolism at the whole organism level farther away from the site of production (insulin from the islets of pancreas, IGF-1 from the liver). These are well known as key regulators of energy metabolism and growth. In fact, their physiologies as systemic hormones were recognized long before the details of their signaling mechanisms at the cellular level were described. This is why the Insulin and IGF-1s differ from many other regulatory peptides that the peptides are relevant to regulate physiology at both the whole organism level and the cellular level. For example, the epidermal growth factor (EGF) and platelet-derived growth factor (PDGF) are examples of peptides that these have important local regulatory roles at the cellular and the tissue levels but not father from the site. There is little evidence to suggest that circulating levels of these growth factors are physiologically significant. This is the reason our invention with the use of Insulin and IGF-1 topically not only has the local effect. They are absorbed and circulated farther away from the site of application and exerts their therapeutic effects on the rods, cones, and in the retina in the retinitis pigmentosa (Michael Pollak. Insulin and Insulin-Like Growth Factor Signalling in Neoplasia. Nat. Rev Cancer. 2008; 8(12):915-928). Besides, the IGFs may be important: autocrine, paracrine, or endocrine growth factor effects. These factors will help to maintain the integrity of photoreceptors when the insulin is transported to the rods and cones of retina from the conjunctival sac as described in our invention.

Insulin is an anabolic trophic hormone needed for the growth, reproduction, and multiplication of all cells in the body including the healthy vascular endothelium, photoreceptors neurons in the retina (rods and cones), macula, as well as secretory glands of the eye lids including the lacrimal glands (afflicted with Sjogren's syndrome) and entire eye ball and its contents. The corneal and conjunctival cells which the cells may be metaplasic in dry eyes syndrome. Increased cellular metabolic activity induced by insulin enhances the uptake and enhances the action of all therapeutic, pharmaceutical, biochemical, and biological agents or compounds by the cells and inside the cell including the cells responsible or involved in retinitis pigmentosa. Insulin enhances their concentration and effectiveness which insulin has disease curtailing-curing qualities. Once inside the cells; the insulin augments and amplifies the effects of any and all therapeutic agents including the agent proven and/or approved to treat retinitis pigmentosa and restoring their physiological function of the rods.

In our decade of studies, medical practice, and experimentation, we found there is not a single disease except hypoglycemia induced by insulin or otherwise, which a disease cannot be treated using Insulin to enhance the effectiveness of the therapeutic, pharmaceutical, biochemical, and biological agents or compounds including the treatment of retinitis pigmentosa.

In ingenious vitro studies, this has been meticulously and conclusively demonstrated that the insulin activates and modifies metabolic pathways in MCF-7 human breast cancer cells. The insulin increases the cytotoxic effect of methotrexate up to 10,000 (ten thousand) fold (Oliver Alabaster' et al. Metabolic Modification by Insulin Enhances Methotrexate Cytotoxicity in MCF-7 Human Breast Cancer Cells, Eur j Cancer Clinic; 1981, Vol 17, ppl 223-1228. Richard L. Schilsky and Frederick. S. Ordway. Insulin effects on methotrexate polyglutamate synthesis and enzyme binding in cultured human breast cancer cells. Cancer Chemother Pharmacol (1985) 15: 272-277). The data suggests that insulin augmentation of MTX polyglutamate synthesis may account for insulin's′ previously observed ability to enhance MTX Cytotoxicity (research studies in human breast cancer). My own research studies on every kind of cancer and infection in any part of the body have shown that the group treated with insulin, plus, with low dose methotrexate and other anticancer agents (and/or antibiotics for infection, autoimmune diseases treatments, monoclonal antibody treatment etc.) responded better than the patient treated with insulin or chemotherapy alone (Eduardo Lasalvia-Prisco et al. Insulin-induced enhancement of antitumoral response to methotrexate in breast cancer patients. Cancer Chemother Pharmacol (2004) 53: 220-224. Ayre S G, Perez Garcia y Belton D, Perez Garcia D jr (1990) Neoadjuvant low-dose chemotherapy with Insulin in breast carcinomas. Eur j Cancer 26:1262-1263; T. R. Shantha presented at Cancun IPT meeting 2nd meeting 2004 and unpublished studies). These observations supports the findings of Alabastor (IBID) that the disease or the healthy cell sensitivity to the therapeutic and biological agents as those to be used to treat retinitis pigmentosa. This can be increased (augmentation/amplification effects) many times by using the method described in this invention using insulin and/or IGF-I. The effect of insulin in reducing the ROS and other etiological factors in retinitis pigmentosa is profound.

Our study of injecting Insulin followed by anticancer chemotherapeutic agents directly into cancer masses on hundreds of advanced and localized cancers supports these finding also. Using this method, the palpable tumors including enlarged lymph nodes with tumors or tumor deposits literally disappeared. We treated multiple brain cancer patients by directly injecting insulin with mannitol followed with specific anti tumor chemotherapeutic agents with dextrose where heparin was directly infused into the internal carotid artery with positive results. Patients lived longer with a good quality of life with fewer side effects to the chemotherapy agents.

We have used insulin locally as a therapeutic agent in chronic non-healing wounds, burns, after draining the hydrocele of the tunica virginals sac in the scrotum, periodontal diseases, post surgical wound healing, delayed healing of broken bones; prostate and bladder afflictions, teeth and gum afflictions, eye and ear diseases and many other diseases; which will be reported later.

The present inventors have used insulin mixed injectate to augment the local anesthetic, or narcotic or steroid effects alone or in combination of the selected therapeutic agents where the agents were introduced into the epidural or subarachnoid space for the treatment of back pain and/or to relieve other kinds of pain due to different etiologies including post operative and cancer pain with excellent rapid, prolonged pain relief (under study).

The present inventors used insulin locally in intravenous regional anesthesia (Bier Block) for surgical procedures of the limbs, pain, to treat reflex sympathetic dystrophy (RDS) and complex regional pain syndrome (CRPS) mixed with ketamine, insulin and known selected therapeutic agents. Previously, the other methods to treat RSD have documented with partial success with injectates containing lidocaine, solumedrol, bretylium, guanethidine, reserpine, ketorolac, and non-steroidal anti-inflammatory drugs in saline (Neil Roy Connellya, Scott Reubena and Sorin J. Brullb Y. Intravenous Regional Anesthesia with Ketorolac-Lidocaine for the Management of sympathetically-Mediated Pain. Yale Journal of Biology and Medicine 68 (1995), pp. 95-99). We had better success using insulin containing injectates with ketamine with above therapeutic agent's solutions in addition to the injectates which this will be reported at a later date. We had better success using insulin with ketamine delivered directly to the CNS in curtailing and curing complex regional pain syndromes (CRPS); reflex sympathetic dystrophy (RSD) & causalgia, phantom leg syndrome, and many pain related complex neurological disorders.

The word, Prolotherapy, means “PROLO” is short for proliferation, because the treatment causes the proliferation (growth and formation) of new ligament tissue (fibroblasts and collagen formation in the weak, stretched or torn ligaments) in areas where the tissue has become weak which the weakness resulted in pain with movement (Ross A. Hauser, Marion A. Hauser. 2007. Prolo Your Pain Away! Curing Chronic Pain with Prolotherapy. Chicago—Amazon books). Many solutions are used in inducing ligamenotous growth such as, dextrose (10%-25%) with lidocaine (a local anesthetic 0.1-0.2%), phenol, glycerin, cod liver oil extract; solution containing 1.25% phenol, 12.5% dextrose and 12.5% glycerin; Glucose 20% and Lidocaine 0.1% solution; mixture of 1 cc of 5% sodium morrhuate and 1 cc of 1% lidocaine; Hackett-Hemwall prolotherapy method of using 15% dextrose, 10% Sarapin (a pitcher plant derivative) and 0.2% procaine solution; or Dr. DeHaan's “Prolo Cocktail” containing 25% of each of the following substances: 50% dextrose, 2% lidocaine or procaine (without epinephrine), vitamin B12 (1000 mcg/ml), and Biosode (“a homeopathic with growth and Krebs cycle energy factors”) has been used.

The inventors have used glucose along with insulin, deferoxamine, and lidocaine in prolotherapy injectate for various musculoskeletal pain, including arthritis, back pain, neck pain, fibromyalgia, sports injuries, unresolved whiplash injuries, carpal tunnel syndrome, chronic tendonitis, partially torn tendons, ligaments, and cartilage, degenerated or herniated discs, TMJ pain and sciatica. It is important to note that the principle of prolotherapy is to induce fibroblasts to multiply and to lay more ligaments (collagen); make the ligaments and tendons stronger inducing sterile inflammation at the site. Insulin enhances the multiplication of fibroblasts; deferoxamine enhances the angiogenesis to support the multiplication of fibroblasts, glucose causes sterile inflammatory response, and the lidocaine alleviates the pain of the injection.

This combination contributes to therapeutic effect of prolotherapy to make the ligaments stronger and pain free. The insulin used in the above preparation which the prolotherapy was more effective compared to when the prolotherapy therapeutic agent was used without insulin. Insulin increased the fibroblast mitosis, thus, increased production of collagen and maintained the integrity of cartilages within the joints to strengthen the ligaments of the painful joint. This gave long lasting rapid pain relief with stronger functional joints when insulin is therapeutically effective in taking away the pain by various prolotherapy therapeutic agents. One can see the effectiveness of the agents in treating the retinitis pigmentosa and associated diseases of the eye. Besides the insulin, purified platelet growth factor added can promote angiogenesis, increased the blood supply, increased the multiplication of fibroblasts on the ligaments, and torn meniscus, and enhanced the healing process.

The purified genetically engineered platelet growth factors are available in the market which can be used to enhance the healing in non-healing bone fractures which the factors can be used to treat the torn meniscus, cartilages and ligaments. Deferoxamine (DFO) is another iron-chelating agent on the formulary that DFO has been shown to increase angiogenesis. We have used Deferoxamine (iron chelator and angiogenesis growth factor, similar to platelet growth factor) and insulin (stimulates metabolic activity and multiplication of cells) sprayed on non healing chronic ulcers with good successes. We have used insulin and Deferoxamine with prolotherapy agents in selected case of ligament tear with joint pain with success. We suspected that some of these cases had meniscus tears. This gave good post therapy results after injecting these therapeutic agents inside the joints besides the collateral ligaments with the avoidance of the surgical intervention.

Trigger points or trigger sites are described as hyperirritable spots in skeletal muscle that are associated with palpable nodules in taut bands of muscle fibers where the compression of the fibers or the application of pressure or the contraction of the muscle where the contraction may elicit local tenderness, referred pain, or local twitch response. There are many therapies to take away the tenderness and the sore spots. Various injections can be used including saline, local anesthetics such as procaine hydrochloride (Novocain); a mixture of lidocaine, and marcaine without steroids (Steroids can cause muscle damage; hence contraindicated) when this is used to relive the pain. Trigger pain point injection for myofacial pain, fibromyalgia, tennis elbow, intercostal pain, wrist and back pains, and injection of joints with therapeutic agents such as local anesthetic with insulin resulted in rapid and effective relief of pain compared to injectate with absence of the insulin. The palpable nodule of trigger point were reduced or disappeared. The same methods can be used to treat the retinitis pigmentosa, and any condition contributing to the retinitis pigmentosa of the eye in combination with other known therapeutic, pharmaceutical, biochemical, and biological agents or compounds as described above.

The examples described above show the effectiveness of the insulin in treating locally disease-afflicted tissue. The same time exert augmentation/amplification effects of therapeutic agents to prevent, delay, curtail and cure the diseases, which the insulin will have the same type of effect in treating retinitis pigmentosa.

In an important experiment, Zheng et al showed the role of insulin like growth factor-I (IGF-I) that insulin like effects induced the inner ear epithelial cell culture growth (Zheng, J. L., Helbig, C. & Gao, W-Q. Induction of cell proliferation by fibroblast and insulin-like growth factors in pure rat inner ear epithelial cell cultures. J. Neurosci. 17:216-226 (1997). There is a clear indication that insulin and IGF-I not only played a role in potentiation of (augmentation/amplification effects) the therapeutic, pharmaceutical, biochemical and biological agents or compounds. They can independently stimulate cells growth in eye structures (as it happens in the inner ear epithelial cells) including retinal cells particularly photoreceptor cells (Shantha T. R., Unknown Health Risks of Inhaled Insulin. Life Extension, September 2007 pages 74-79, Post publication comments in September 2008 issue of Life Extension, Pages 24. Shantha T. R and Jessica G. Inhalation Insulin, Oral and Nasal Insulin Sprays for Diabetics: Panacea or Evolving Future Health Disaster. Part I: Townsend Letter Journal: Issue #305, December 2008 pages: 94-98; Part II: Townsend Letter, January, 2009, Issue # 306, pages-106-110).

The normal cell undergoes the following changes as pathological state takes its root:

1. Dysplasia, where cell maturation and differentiation are delayed, often indicative of an early neoplastic process. The term dysplasia is typically used when the cellular abnormality is restricted to the originating tissue, as in the case of an early, in-situ neoplasm. This means that the original cells are not robust enough to withstand the new environment. The cells changes into another type more suited to the new environment.

2. Metaplasia is the reversible replacement of one differentiated cell type with another mature differentiated cell type. The medical significance of metaplasia is in some sites. The cells may progress from metaplasia, to develop dysplasia, and then malignant neoplasia (cancer).

3. This is contrasted with heteroplasia, which heteroplasia is the abnormal growth of cytological and histological elements without a stimulus. Insulin has profound effect on these cells undergoing metaplasia and dysplasia. Heteroplasia is indicated in our above articles published in Life Extension and Townsend letters research publications; The changes contributing to the pathology of the eye diseases including retinitis pigmentosa whose progression halted and reversed; restore normal functioning by insulin alone or combined with insulin and other known therapeutic agents to treat retinitis pigmentosa.

Insulin exerts the trophic effect on the cell physiology without discriminating whether it is normal, metaplasic, dysplasic, heteroplasic, or carcinogenic (Philpott M P, Sanders D A, Kealey T. Effects of insulin and insulin-like growth factors on cultured human hair follicles: IGF-I at physiologic. J Invest Dermatol 1994; 102: 857-61, Shantha IBID). This is a known physiological phenomenon that the insulin does bind to the receptor sites of the IGF-I and insulin. The insulin exerts multiple profound physiological and pharmacological therapeutic effects. The insulin induces cell growth (besides glucose transport) enhances the metabolism, and increases the glutathione needed for the cells' health. This enhances mitosis and increases the production of nuclear proteins in the nucleus and ribonucleoprotein production by the endoplasmic reticulum, activates the Golgi complex, and enhances the lysosomes activity.

Thus, the insulin helps to break up endocytosed materials and cellular debris to eliminate the cellular toxins which the insulin enhances (augmentation/amplification effects) the therapeutic effect of other pharmacological agents (Shantha T. R., Life Extension September 2007:74-79,) where insulin binds on the cell. This has been reported in the above publications. Thus, any dysfunction of the retina seen in retinitis pigmentosa will be restored back to normal using the described inventive methods. The present eye drops used to treat the retinitis pigmentosa don't contain therapeutic agents to repair and to restore the damaged or disease afflicted rods. The tissues involved where the body uses its own physiological hormone locally as described in our invention.

Insulin, potassium, and glucose are routinely administered to treat low potassium levels in the cells even to this day. The inventor has used this method to lower the potassium levels in the blood for more than 3 decades. Insulin and glucose facilitates the entry of potassium inside the cell—a life saving measure. Similarly, the Insulin deposited in the conjunctival sac will enhance the uptake of pharmaceutical, biochemical, nurticeuticals and biological agents or compounds, as well as drugs and therapeutic agents by the dysfunctional cells of the retina, reduces the ROS to prevent further damage to the rods (cones) and to restore the function of the retina described in this inventive method to treat RP.

The inventors have used insulin as potentiator of uptake and enhancer of therapeutic action of diverse therapeutic agents to cure and/or curtail curable acute, chronic, and incurable diseases such as cancer, Lyme disease, scleroderma, lupus, psoriasis, antibiotic resistant staphylococcus infection (MRSA infection), chronic wounds, neurological diseases, inner and middle ear affliction, autoimmune diseases, leprosy, prostate pathologies, skin diseases, herpes zoster of the eye with antiviral agents and tuberculosis. Many other diseases have had good results with the method. Inventors have used insulin with other specific treatment modalities against depression, Alzheimer's, Autism, Parkinson's, and many other neurological diseases successfully. The insulin needs to be delivered to the brain through proper routes where the routes are going to be reported in later publications which we described in our utility patent application and on the rabies cure presentations (T. R. Shantha. RABIES CURE. Ffs ID# 8527859, U.S. patent application Ser. No. 12/893,827, confirmation # 4848, Shantha, T. R. Site Of Entry Of Rabies Virus Form The Nose And Oral Cavity; And New Method Of Treatment Using Olfactory Mucosa And By Breaking BBB, presented at The 2nd International Rabies In Asia Conference Held In Hanoi, 2009, Pp 70-73, and The Rabies in the North Americus (XX RITA), held in Quebec City, 2009, Pp 20-21, Rabies cure, patent pending 2009.).

The present inventors have used insulin for more than a decade to enhance the effectiveness of locally injected therapeutic agents especially cancers with chemotherapeutic agents with remarkable results. Our data supports that the insulin sprayed on indolent ulcers anywhere in the body, including the oral (gums), and the nasal cavity augmented the healing. Insulin stimulated the fibroblast, endothelial cell, angiogenesis, and skin cell growth resulting in accelerated wound healing. Application of insulin soaked cotton swabs (1-3 units in normal saline) after teeth extraction induces rapid healing with reduced pain. Studies show that the application of insulin and antibiotics locally on the gums with or without Xylitol eliminated gum diseases (periodontitis), made the loose teeth firm, reduced the tooth caries, cleared the root infection rapidly with dental practices which the dental practices are under study (Dr. Hughes, J. DDS: Personal communication).

Insulin is a metabolic activity enhancer of all cells and therapeutic agents. Insulin can play an important role in treatment of many diseases including retinitis pigmentosa by increasing the metabolic activity, protecting against ROS damage, and preventing further degeneration of rod and cone segments and restoring the mitochondrial function (Shantha T. R.; 1. discovery of insulin and IPT: amazing history, 2. high dose methotrexate therapy using Insulin; 3. local injections of tumors with insulin and cytotoxic drugs; 4. two and three cycle insulin Potentiation therapy: Presented at 2nd international conference on Insulin Potentiation Therapy held at Cancun, Mexico, Jun. 28-Jul.1, 2004).

A synergy between certain membranes and metabolic effects of insulin on cell molecular biology increases therapeutic efficacy of all anti retinitis pigmentosa therapeutic, pharmaceutical, biochemical, and biological agents or compounds which the insulin reduces doses of the drugs, enhancing their uptake with augmentation/amplification effects greater than before the therapeutic efficacy. The insulin enters the cells where the insulin increases the effectiveness of therapeutic agents many properties. Thus, the present inventive method not only enhances the uptake of therapeutic agents, but, the insulin enhances their therapeutic effect inside the cells of the disease afflicted cells as reported by Alabaster (IBID).

It is known that the pharmaceutically acceptable oxidizing agent facilitates the delivery of the bioactive agent through the skin and mucous membranes which the membranes includes the oral cavity, nasal passages, and conjunctiva. In general, the oxidizing agent can react with molecules present in the conjunctiva where a reaction of adversity with the bioactive agent. For example, the reduction of the glutathione which glutathione is present in the mucus membranes and the skin can inactivate bioactive agents such as insulin by breaking chemical molecular bonds. Not wishing to be bound by theory, when delivering insulin through the skin and mucous membranes, reduced glutathione that it can inactivate insulin. Specifically, insulin has numerous disulfide bonds which are crucial for the protein conformation, biological activity, and subsequent therapeutic effects. Reduced glutathione will inactivate insulin by reducing or breaking insulin's disulfide bonds. Once these disulfide bonds are broken; insulin becomes inactive due to lost protein conformation and biological activity. Thus, the administration of the oxidant by eye drops (as described by Shantha et al in U.S. Patent Application Pub. No. 2009/0347776 A1) herein, prevents the inactivation of the bioactive agent such as insulin when applied to the skin, mucus membrane, and conjunctival sac of the eye. Specifically, application of an oxidant or a pharmaceutically oxidizing agent to conjunctival sac will lower or prevent the effects of reduced proteins. The reduction of the biological molecules has on the bioactive agents which the inactivation of bioactive agents via reduction or cleavage of crucial molecular bonds will be avoided.

The selection and the amount of the pharmaceutically acceptable oxidizing agent can vary depending upon the bioactive agent that agent is to be administered. In one aspect, the oxidizing agent includes, but is not limited to iodine, povidone-iodine, and any source of iodine or combinations of oxidants, silver protein, active oxygen, potassium permanganate, hydrogen peroxide, sulfonamides, dimethyl sulfoxide or any combination thereof. These oxidizing agents may act as absorption agents which the oxidizing agents help facilitate delivery of a therapeutic agent onto and into the skin. In one aspect, the oxidant is at least greater than 1% weight per volume, weight per weight, or mole percent.

Our preliminary studies have shown that the conjunctiva unlike normal skin and other mucus membranes don't act as a barrier like stratum corneum of the skin for entry of insulin due to the paucity of the presence of reduced glutathione. The conjunctiva doesn't contain any insulin blocking agent, besides conjunctiva doesn't have the multilayered stratum coneium as seen on the skin. This blocks the entry of insulin from the skin. The insulin deposited in the conjunctival sac is rapidly absorbed by the conjunctiva, cornea, and bulbar conjunctiva, retina, choroid, ciliary body and processes, iris, anterior and posterior chambers of the eye, retro bulbar space and helps the entire retina including the photoreceptors to recover from retinitis pigmentosa affliction and any pathological state affecting the vision. The insulin prevents the progression of retinitis pigmentosa.

In one aspect, transconjunctival penetration of insulin and known therapeutic agents, as well as other pharmaceutical, biochemical, nurticeuticals and biological agents or compounds can be facilitated by enhancers; the enhancers can be used to further expedite the entry of these agents to penetrate and to permeate inside the eye ball where the agents are delivered to choroid and retina. Penetration enhancers not only penetrate a membrane efficiently but these enhancers enable other bioactive agents to cross a particular membrane more efficiently. Penetration enhancers produce their effect by various modalities such as disrupting the cellular layers of the conjunctival sac surface interacting with intracellular proteins and lipids, or improving partitioning of bioactive agents as they come into contact with the mucosal membranes.

The entry into BV and Lymphatics of the eye which the BV dissipates them to the contents of the eye ball within the retina. These enhancers, macromolecules up to 10 kDa are able to pass through the conjunctival sac layers of the eyes where they reach the site of retinitis pigmentosa which the blood vessels, RPE and retina are undergoing pathological changes. These enhancers should be non-toxic, pharmacologically inert, non-allergic substances.

In general, these enhancers may include anionic surfactants, urea's, fatty acids, fatty alcohols, terpenes, cationic surfactants, nonionic surfactants, zwitterionic surfactants, polyols, amides, lactam, acetone, alcohols, and sugars. In one aspect, the 10 penetration enhancer includes dialkyl sulfoxides such as dimethyl sulfoxide (DMSO), decyl methyl sulfoxide, dodecyl dimethyl phosphine oxide, octyl methyl sulfoxide, nonyl methyl sulfoxide, undecyl methyl sulfoxide, sodium dodecyl sulfate and phenyl piperazine, or any combination thereof. In another aspect, the penetration enhancer may include lauryl alcohol, diisopropyl sebacate, oleyl alcohol, diethyl sebacate, dioctyl sebacate, dioctyl azelate, hexyl laurate, ethyl caprate, butyl stearate, dibutyl sebacate, dioctyl adipate, propylene glycol dipelargonate, ethyl laurate, butyl laurate, ethyl myristate, butyl myristate, isopropyl palmitate, isopropyl isostearate, 2-ethylhexyl pelargonate, butyl benzoate, benzyl benzoate, benzyl salicylate, dibutyl phthalate, or any combination thereof which are opthalmologically acceptable to be used for local instillation.

In other aspects, these additional components with insulin may include antiseptics, antibiotics, anti-virals, anti-fungals, anti-inflammatories, anti-dolorosa, antihistamines, steroids, vasodilators and/or vasoconstrictors to reduce inflammation, irritation, or reduce rapid absorption through conjunctival sac. Such vasoconstrictors may include phenylephrine, ephedrine sulfate, epinephrine, naphazoline, neosynephrine, vasoxyl, oxyrnetazoline, or any 5 combinations thereof.

Such anti-inflammatories may include non-steroidal anti-inflammatory drugs (NSAIDs). NSAIDs alleviate pain and inflammation by counteracting Cyclooxygenase and preventing the synthesis of prostaglandins. In one aspect, NSAIDs include celecoxib, meloxicam, nabumetone, piroxicam, napmxen, oxaprozin, rofecoxib, sulindac, ketoprofen, valdewxid, anti-tumor necrosis factors, 10 anti-cytokines, anti-inflammatory pain causing bradykinins or any combination, thereof. Such antiseptics, anti-virals, anti-fungals, and antibiotics, may include ethanol, propanol, isopropanol, or any combination thereof; a quaternary ammonium compounds including, but not limited to, benzalkonium chloride, cetyltrimethylammonium bromide, cetylpyridinium chloride, benzethonium chloride, or any combination thereof; boric acid; chlorhexidine gluconate, hydrogen peroxide, iodine, mercurochrome, ocetnidine dihydrochloride, sodium chloride, sodium hypochlorite, silver nitrate, colloidal silver, mupirocin, erthromycin, clindamycin, gentamicin, polymyxin, bacitracin, silver, sulfadiazine, or any combination thereof. The intention of this invention is to use insulin with the above described anti-inflammatory and antibacterial agents. These can eliminate the pathogenic factors contributing to the retinitis pigmentosa and to restore normal sight.

In accordance, with one aspect of the invention, the compounds used to apply locally to the eyes site are mixed which the conjunctiva is a suited vehicle or carrier. The compositions of this invention may comprise aqueous solutions such as e.g., physiological saline, oil, gels, patches, solutions or ointments. The vehicles which carry these biologically active therapeutic agents may contain conjunctivally compatible preservatives such as e.g., benzalkonium chloride, surfactants like e.g., polysorbate 80, liposome's or polymers: examples like methyl cellulose, polyvinyl alcohol, polyvinyl pyrrolidone, and hyaluronic acid and others. Sterile water or normal saline are used in some of the preparations of the eye drops for our invention.

There are various forms of insulin used to treat diabetes which different forms of insulin can be formulated to be used in this invention. They are grouped under rapid, short, intermediate, and long acting insulin. The insulin is dispensed as premixed form containing rapid to long acting insulin. Insulin products are categorized according to their putative action (see Table IV) profiles as:

1. Rapid-acting: insulin lispro, l. aspart, and l. glulisine

2. Short-acting: regular (soluble) insulin

3. Intermediate-acting: NPH (isophane) insulin

4. Long-acting: insulin glargine and insulin detemir

The table I summarizes: the time of onset; peak action and duration of action of the different types and the different brands of insulin that the insulin can be used in our invention.

| | | | | |
|---|---|---|---|---|
| Insulin Preparation | Onset of | Peak | Effective | Maximum |
| and their generic and | Action in | action in | duration of | duration in |
| trade names | hours | hours (h) | action (h) | hours |

RAPID-ACTING INSULIN ANALOGUES AND PREPARATIONS

| | | | | |
|---|---|---|---|---|
| Insulin lispro | ¼-½ | ½-1¼ | 3-4 | 4-6 |
| (Humalog), Insulin | | | | |
| aspart (NovoLog), | | | | |
| Insulin glulisine | | | | |
| (Apidra) | | | | |

SHORT-ACTING INSULIN

| | | | | |
|---|---|---|---|---|
| Regular (soluble) | ½-1 | 2-3 | 3-6 | 6-8 |

INTERMEDIATE-ACTING

| | | | | |
|---|---|---|---|---|
| NPH (isophane) | 2-4 | 6-10 | 10-16 | 14-18 |

LONG-ACTING INSULIN ANALOGUES

| | | | | |
|---|---|---|---|---|
| Insulin glargine | 3-4 | 8-16 | 18-20 | 20-24 |
| (Lantus) | | | | |
| Insulin detemir | 3-4 | 6-8 | 14 | ~20 |
| (Levemir) | | | | |

Glucose concentrations are expressed as milligrams per deciliter (mg/dL or mg/100 mL) in the United States, japan, Spain, France, Belgium, Egypt, and Colombia. The millimoles per liter (mmol/L or mM) are the units used in the rest of the world. Glucose concentrations expressed as mg/dL can be converted to mmol/L by dividing by 18.0 g/dmol (the molar mass of glucose). For example, a glucose concentration of 90 mg/dL is 5.0 mmol/L or 5.0 mM. During a 24 hour period blood plasma glucose levels are typically between 4-8 mmol/L (72 and 144 mg/dL). Although, 3.3 or 3.9 mmol/L (60 or 70 mg/dL) is referred to as the lower limit of normal glucose. The symptoms of hypoglycemia typically do not occur until 2.8 to 3.0 mmol/L (50 to 54 mg/dl). The precise level of glucose considered low enough to define hypoglycemia is dependent on (1) the measurement method, (2) the age of the person, (3) presence or absence of effects (symptoms), and (4) the purpose of the definition. The debate continues to what degree of hypoglycemia warrants medical evaluation or treatment, or can cause harm.

One has to realize the possibility of developing hypoglycemia when the insulin is being used as ophthalmic drops due to nasal mucosal absorption draining through the nasolacrimal ducts (FIG. 3). Patients will be warned about the possibility of hypoglycemia which they will be prepared for a hypoglycemic reaction. FIG. 3 shows the prevention of the drainage to the nose. In our practice of using the insulin eye drops for decades, we never reported the development of a single case of hypoglycemic effects using the ophthalmic insulin drops used in our study. The typical threshold for hypoglycemia is 70 mg/dL (blood sugar level of 3.9 mmol/L), although, hypoglycemia may be higher or lower depending on a patient's individual blood glucose target range. Generally, the hypoglycemia is defined as a serum glucose level (the amount of sugar or glucose in a person's blood) below 70 mg/dL. Symptoms of hypoglycemia, in general, appear at levels below 60 mg/dL. Some people may experience symptoms above this level. Levels below 50 mg/dL affect the brain function. Signs and symptoms of hypoglycemia include erratic or rapid heartbeat, sweating, dizziness, confusion, unexplained fatigue, shakiness, hunger, feeling hot, difficulty in thinking, and headache. Some may be even develop seizures and potential loss of consciousness with severe hypoglycemia. Once symptoms of hypoglycemia develop, the patient should be treated with oral ingestion of a fast-acting carbohydrate such as glucose tablets, fruit juice, fruit bowl, chocolate bar, or regular Coca-Cola, sugary drinks or eat plain sugar followed with a drink of water or IV administration of 25% glucose if there is severe hypoglycemic which the patient has an IV established. There is importance to test the blood sugar 15 minutes after administration if symptoms of hypoglycemia develop with a finger stick sugar tester strips. It has been projected that the newborn brains are able to use alternate fuels when glucose levels are low more readily than adults.

Preparation of the Retinitis Pigmentosa Patients for Therapy Using Our Inventive Method of Using Insulin

Before using described inventive methods and examples; a thorough examination of the affected patient's eye is in order. The examination of the eye may include: 1. Acuity testing, 2. Biomicroscopy, 3. intraocular pressure (IOP), 4. Opthalmoscopy, 5. Color vision test, 6. Tear osmolality, 7. Schimer's test, 8. Tear film breakup time (tBUT), 9. Test for Superficial punctate keratitis (SPK), 10. Fluorescein and Rose Bengal staining (RBS) of BV of the retina, as well as cornea, conjunctiva, and eyelids, 11, slit-lamp examination of the conjunctiva, cornea, anterior chamber, iris, and lens, 12. The Ocular Surface Disease Index (OSDI), 13. Microscopic examination of the tear filament, 14. Maturation index (a Papanicolaous stained sample of conjunctival epithelium). 15. The most important test for retinitis pigmentosa is electroretinogram (ERG) to measure the function of the photoreceptors. In addition, a complete physical examination with blood test for thyroid, parathyroid, growth hormone, insulin, IGF-1, FSH, LH, cortisol, estradiol, and testosterone levels, electrolytes, blood cell count, cholesterol levels, ESR, and a urine sample for pregnancy test when this is deemed necessary when the patient is of childbearing age.

To apply our inventive ophthalmic insulin drops as therapeutic agents, the patient or the care giver will wash their hands with mild antiseptic soap. The person or patient applying the drops must be careful not to touch the dropper tip to the eye lids (and the foreign objects) to avoid contamination if there is an eye lid infection. Tilt the head back, or lay down with head extended on a neck pillow; gaze upward and backwards, and pull down the lower eyelid to expose the conjunctival formix. Place the dropper directly over the eye away from the cornea and instill the prescribed number of drops. Look downward and gently close your eye for 1 to 2 minutes. The patient should not rub the eye. Do not rinse the dropper unless the patient or person knows the sterilization technique with hot water.

If other therapeutic, pharmaceutical, biochemical and biological agents or compounds are to be selected to treat the condition with our invention; wait at least 3-5 minutes before using other selected anti-retinitis pigmentosa therapeutic agents or the other variety of ophthalmic medicaments. It is important to instill medications regularly as prescribed to control retinitis pigmentosa. Consult your doctor and/or pharmacist if the systemic medications that you are taking are safe to use with the eye drops described and prescribed. When there is no contraindication for the insulin eye drops, you can treat patients, except, the patients with hypoglycemia syndromes and in some cases external ocular tumors.

To minimize the absorption into the bloodstream and to maximize, the amount of drug absorbed by the eye, close your eye for one to five minutes after administering the insulin drops. Then, press your index finger gently against the inferior nasal corner of your eyelid to close the tear duct which drains into the nose (FIG. 6). This will prevent any adverse systemic effects due to nasal vascular uptake into the systemic circulation from the nasolacrimal duct drainage of the therapeutic agents from the conjunctival sac. Eye drops may cause a mild uncomfortable burning or light stinging sensation which this reaction should last for only a few seconds.

The anti-retinitis pigmentosa drops take effect after 5-10 minutes after application depending upon the therapeutic agents used with the eye drops. We recommend best to use insulin eye drops before bed time and rising in the morning. This process can be repeated every 6-12 or 24 hourly for 3-7 days a week till the desirable results are obtained. Retinitis pigmentosa patients can use insulin eye drops all their lives or intermittently, depending on the results and the need. The therapeutic agents are instilled using a sterile dropper (or bottle with medication equipped with a dropper nipple) into the conjunctival sac.

Experiments by the present inventors has shown that the local application of rapid action or other types of insulin formulations on the balding scalp, eye lid hair line, on the gums, oral and nasal mucosa, and conjunctival sac, surgical wounds, open area of extracted wisdom teeth, local injections of tumors, injection into tunica vaginalis testes, other regional and local sites did not change the blood sugar levels (without hypoglycemic effects) indicates, that there is safety to use up to 1-2-3 IU (international units) insulin to the conjunctival sac of both eyes without hypoglycemia effects. The present invention formulations contain only 0.10 to 1.00 IU per drop which the dosage can be increased or the dosage can be decreased depending upon the disease states.

Preparation of Insulin Eye Drops for Use in Retinitis Pigmentosa

Take 100 international units (IU) of rapid or intermediate or long acting insulin (or IGF-1) and dilute in 5 ml of sterile saline or distilled water or other carriers and facilitators as described above. The pH can be adjusted to prevent the sting when the insulin is dropped into the conjunctival sac. The preparation can contain nanograms (micrograms) of local anesthetics to prevent the stinging when the eye drops are applied to the eye. In this preparation, each ml contains 20 units of insulin. In pharmacies, a drop was another name for a minim, which a drop would be 0.0616 milliliters. The drop is standardized in the metric system to equal exactly 0.05 milliliters. The 20 drops equal one ml (l cc) which each drop contains 0.10 IU of insulin.

The concentration of the insulin content can be increased to 0.20, 0.30, 0.40, and 0.50 or even up to 1 or 2 unit of insulin per drop. The insulin content can be increased per drop in the dilutant preparation. The insulin content can be decreased by reducing the insulin units used for the preparation of the ophthalmic drops. Instill one to two drops to each eye lower lid formix and/or everted upper eyelid (conjunctival sac) as a single agent. The applicant must apply pressure on the nasolacrimal duct as shown in the FIG. 6 to prevent drainage into the nasal cavity.

If other combinations of the anti-retinitis pigmentosa therapeutic agents are to be used: first use insulin drops, wait for 3-5-10 minutes and apply the other therapeutic, pharmaceutical, biochemical, and biological agents or compounds. After this procedure, instill one more insulin drop to further enhance the uptake of the other selected therapeutic agents to augment-amplify their effects at the cellular level. This step is optional and may not be needed in most of the cases. The dose used in our invention can be appropriately selected depending upon symptom, age, severity of the disease, dosage form, and existing health conditions. The pH can be within a range which the pH is acceptable to ophthalmic preparations which the pH preferably is within a range from 4-6-7 to 8 most preferably 7.4.

The data supports the other therapeutic agents which the agents are used after insulin where the agents are prepared in 5-10% solutions of glucose. The glucose acts as a carrier of the therapeutic agents after pretreatment with insulin. I have named this local Insulin Potentiation Therapy (LIPT).

Insulin can be compounded as a liquid ophthalmic isotonic solution containing cyclosporin, or other antiautoimmune therapy agents, or vitamins, and one or more one buffering agents, said buffering agents producing a pH in said composition similar to mammalian eye fluids.

The insulin pharmaceutical eye drop preparation of this invention may contain 0.25%-0.5%-1%-2% or more glucose. There are several mechanisms which glucose and insulin protect the damaged cells that the insulin restores normal function. Glucose is the preferred substrate during periods of cell damage and ischemia. Adenosine triphosphate derived from glycolysis is vital for stabilization of membrane ion transport which electroporation, iontophoresis, sonophoresis, vibroacoustic and vibration methods transport can enhance. This biological activity is enhanced by insulin.

This is crucial to the above biological activity needed for cellular integrity, endothelium, vascular smooth muscle cells, and nerve cells like the retina, photoreceptors and their synapses. Preservation of these functions in these structures of the eye especially the retina decreases any further damage and participates in the repair. Glucose esterifies intracellular free fatty acids, which these decreases their toxic end-products and oxygen free radicals. Glucose is a direct precursor of pyruvate, which pyruvate is carboxylated to the citric acid cycle substrates malate and oxaloacetate which this can replenish depleted substrates, thus, stimulating oxidative aerobic metabolism, reduce the ROS production and their adverse effect on photoreceptors. Glucose with the help of insulin esterifies intracellular free fatty acids which the fatty acids decreases their toxic end-products and oxygen free radicals.

Experimental studies have shown that glucose converted to pyruvate with the help of insulin can restore the function through the replenishment of depleted citric acid substrates. This helps in the repair and the restoration of the photoreceptors cellular function. This helps in curtailing or in curing the retinitis pigmentosa. Experiments show that the glucose is converted to pyruvate in the presence of insulin which the insulin can restore contractile function of the blood vessel, various histological components of the retina, choroid and ciliary muscles through the replenishment of depleted citric acid. Thus, our invention with the use of insulin with glucose can help in relieving and reversing the retinitis pigmentosa pathology, signs, symptoms, and restore the physiological state to the pigment epithelial cells.

Insulin stimulates pyruvate dehydrogenase activity, which the activity in turn stimulates aerobic metabolism. Exogenous insulin helps to reverse insulin resistance which this reversal can be of benefit in retinitis pigmentosa associated with diabetes. The importance is the glucose which the insulin facilitates the entry of other therapeutic agents into the normal and disease afflicted cells.

The above pharmaceutical eye drop preparation of our invention may contain antibacterial components which these components are non-injurious to the eye when used. Examples are: thimerosal, benzalkonium chloride, methyl and propyl paraben, benzyldodecinium bromide, benzyl alcohol, or phenyl ethanol. There is an autism controversy which we will avoid using thimerosal.

The therapeutic pharmaceutical preparation may contain buffering ingredients such as sodium chloride, sodium acetate, gluconate buffers, phosphates, bicarbonate, citrate, borate, ACES, BES, BICINE, BIS-Tris, BIS-Tris Propane, HEPES, HEPPS, imidazole, MES, MOPS, PIPES, TAPS, TES, and Tricine.

The therapeutic, pharmaceutical, biochemical, and biological agents or compounds used in our invention may also contain a non-noxious pharmaceutical carrier, or with a non-toxic pharmaceutical inorganic substance. Typical of pharmaceutically acceptable carriers are, for example: water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, olyvinylpyrrolidone, isopropyl myristate and other traditionally acceptable carriers.

The therapeutic preparation may also contain non-toxic emulsifying, preserving, wetting agents, bodying agents, as for example: polyethylene glycols 200, 300, 400 and 600, carbowaxes 1,000, 1,500, 4,000, 6,000 and 10,000, antibacterial components as quaternary ammonium compounds, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic. Furthermore, appropriate ophthalmic vehicles can be used as carrier media for the current purpose, including, a conventional phosphate buffer vehicle systems, isotonic boric acid vehicles, isotonic sodium chloride vehicles, isotonic sodium borate vehicles and the like.

The objects are accomplished by treating the eye with an aqueous composition containing an effective amount of a nonionic surfactant and insulin. The applicant has found that an effective amount of surfactant may comprise anywhere from 0.5 percent by weight and by volume to about 10 percent by weight and volume (hereinafter %), preferably about 1-5%, of active surfactant (not combined with oil) in the composition combined with insulin. However, the use of any oil in the composition will reduce the effectiveness of the surfactant. The reason is that a substantial percentage of the surfactant tends to serve as a vehicle for dissolving or forming an emulsion of the oil with the aqueous layer to “wash” or hydrate the corneal surface. Thus, any oil is used in the composition, then additional surfactant will be required to provide the effective amount of 0.5-10% preferably 1-5%, of available active nonionic surfactant.

The anti-retinitis pigmentosa therapeutic agents preparation may contain surfactants such as polysorbate surfactants, polyoxyethylene surfactants (BASF Cremaphor), phosphonates, saponins, and polyethoxylated castor oils, but preferably the polyethoxylated castor oils are commercially available.

The pharmaceutical preparation may contain wetting agents which the agents are already in use in ophthalmic solutions such as carboxymethylcellulose, hydroxypropyl methylcellulose, glycerin, mannitol, polyvinyl alcohol or hydroxyethylcellulose. The diluting agent may be water, distilled water, sterile water, or artificial tears. The wetting agent is present in an amount of about 0.001% to about 10%.

The ophthalmic formulation of this invention may include acids and bases to adjust the pH; tonicity imparting agents such as sorbitol, glycerin and dextrose; other viscosity imparting agents such as sodium carboxymethylcellulose, polyvinylpyrrdidone, polyvinyl alcohol, and other gums; suitable absorption enhancers, such as surfactants, bile acids; stabilizing agents such as antioxidants, like bisulfites and ascorbates; metal chelating agents, such as sodium EDTA; and drug solubility enhancers, as polyethylene glycols. These additional ingredients help give commercial solutions stability which they don't need to be compounded.

Ophthalmic medications compositions will be formulated as to be compatible with the eye and/or contact lenses. The eye drop preparation should be isotonic with blood. As will be the ophthalmic compositions intended for direct application to the eye will be formulated as to have a pH and tonicity which these are compatible with the eye. This will normally require a buffer to maintain the pH of the composition at or near physiologic pH (i.e., pH 7.4) which the buffer may require a tonicity agent to bring the osmolality of the composition to a level or near 210-320 millimoles per kilogram.

In the following detailed, description of the invention, reference is made to the drawings, microphotographs and tables which reference numerals refers to the like elements which the elements are intended to show by way of illustration specific embodiments where the invention we describe using insulin, and IGF-1 with or without other known anti retinitis pigmentosa therapeutic, pharmaceutical, biochemical, and biological agents or compounds enumerated, They may be prescribed and practiced. This is understood where other embodiments may be utilized that the structural changes may be made without departing from the scope and the spirit of the invention described herein.

The eye drop composition of the invention includes buffering agents to adjust the acidity or the alkalinity of the final preparation to prevent eye irritation. The composition is an isotonic solution in that it has the similar pH to fluids indicating that the pH of the composition is 6.1, 6.3, or 7.4. The buffering agents may include all of zinc sulfate, boric acid, and potassium necessary to be effective in achieving the pH of the composition of from 6.10 to 6.30, and to 8.00 typically. The total amount of buffering agents present in the composition ranges from 1% to 10% by weight of the composition.

The eye drop composition includes a lubricant such as cellulose derivatives (carboxymethyl cellulose). The composition may contain known preservatives conventionally used in eye drops such as benzalkonium chloride and other quaternary ammonium preservative agents, phenyl mercuric salts, sorbic acid, chlorobutanol, disodium edentate (EDTA), thimerosal, methyl and propyl paraben, benzyl alcohol, and phenyl ethanol. Purified benzyl alcohol may be in the concentration preferably from 0.1% to 5% by weight.

The eye treatment composition of the invention is a solution having a vehicle of water or mixtures of water and water-miscible solvents. For example, lower alkanols or arylaikanols, the phosphate buffers vehicle systems and isotonic vehicles where the vehicles are boric acid, sodium chloride, sodium citrate, sodium acetate and the like, vegetable oils, polyalkylene glycols, and petroleum based jelly, as well as aqueous solutions containing ethyl cellulose, carboxymethyl cellulose, and derivatives thereof. The hydroxypropylmethyl cellulose, hydroxyethyl cellulose, carbopol, polyvinyl alcohol, polyvinyl pyrrolidone, isopropyl myristate, and other conventionally-employed non-toxic, pharmaceutically acceptable organic and inorganic carriers.

The composition is applied to the eye. The composition should be sterile in the form of an isotonic solution. The constitution may contain non-toxic supplementary substances such as emulsifying agents, wetting agents, bodying agents, and the like. For example, polyethylene glycols, carbowaxes, and polysorbate 80 and other conventional ingredients can be employed such as sorbitan monolaurate, triethanolamine, oleate, polyoxyethylene sorbitan 35 monopalmitylate, dioctyl sodium sulfosuccinate, monothioglycerol, thiosorbitol, ethylenediamine tetraacetic acid, and the like.

Maintenance of Photoreceptors, Muller Cells, and Retinal Pigment Epithelium (RPE) Function by Insulin Ophthalmic Therapeutic Agent of Our Invention

A wide array of blinding and visually impairing disorders including retinitis pigmentosa are caused by degeneration of the photoreceptors of the retina. The retina is a intricate convoluted structure comprising 10 layers of neuronal cell types (FIG. 5), their synapses, and their axons, as well as the complex Muller glial cells, and their arrangement on retinal pigment epithelium (RPE). The health and the continued existence of the photoreceptors are greatly dependent on the integrity of other surrounding cell types of the retina, especially RPE cells and the Muller cells. RPE-secrete proteins including pigment epithelium-derived factor (PEDF) to promote photoreceptor differentiation and survival of the photoreceptor. Our invention with the use of insulin will augment the production of PEDF from the RPE cells to maintain the photoreceptors cells integrity and their physiological state.

The Mulller cells of the retina are recognized to play important roles in photoreceptor development and survival. Muller cells are coupled embryologically, physically, and metabolically to photoreceptors. The Muller cells provides trophic support to promote photoreceptor survival which the survival may regulate synaptogenesis and neuronal processing through bidirectional communication. Delivery of insulin in ophthalmic drops will help to maintain the integrity of Muller cells. This helps to maintain the structure and the function of the photoreceptors which the Muller cells play a role in the treatment of retinitis pigmentosa.

Even now, the mechanisms of how the numerous genetic mutations in the rods of RP patients could give rise to damaging free-radical reactions capable of triggering apoptosis through their adverse effects on mitochondria's and rods outer segment function isn't known at this time. One of the important parts of our invention is to focus on free radical adverse effect of ROS reactions in RP where the invention will provide a rationale simple therapy by use of wide-ranging array of antioxidants and nutritional supplements with insulin for stemming progression of RP.

In particular, our invention focuses on saving photoreceptors not affected by the genetic problems of the rods and cones, which the cells can become lethally damaged by a spill-over of free radicals and related harmful chemical reactions occurring in the rods. Rods, amacrine and horizontal cells of the retina undergo neurite sprouting in human retinas with retinitis pigmentosa. These changes in the retinal neurons may contribute to the electroretinographic abnormalities and the progressive decline in vision noted by patients with retinitis pigmentosa.

Photoreceptors are structurally polarized neurons with one pole of the neurons are the chemical synapses; at the other end is the outer segment, the most highly specialized region of the photoreceptor cells where the Vision originates. Our invention of using insulin will help to maintain the integrity of the retinal pigment epithelium, Muller cells, and the most sensitive parts of photoreceptors (the outer segment with the mitochondria) by providing needed metabolic, nutritional trophic factor support, and by facilitating the removal of the ROS from the site, increasing the output of ATP by using glucose to restore their function and supporting physiological functioning of these three structural units.

This and other metabolic and therapeutic qualities of the insulin will prevent the development, stop the progression, and curtail or cure the retinitis pigmentosa. I have used insulin ophthalmic drops for various oculopathies, including, retinitis pigmentosa for years with great success.

Free Radical Damage in Retinitis Pigmentosa: Our Invention Prevents, Curtails or Cures Free Dadical Damage which are Involved in Retinitis Pigmentosa Development

The pathophysiology of the retinitis pigmentosa isn't known. RP is the result of a defect in the physiological mechanisms of the protection against the photo-oxidative processes involving free radicals (ROS). The retinal degeneration is the result of a deficiency in the protective physiological mechanisms. Our invention encompasses:

1. Protection against the photo-oxidative processes involving free radicals (ROS).

2. Attenuate and ease the biological effects of sun radiations on the retina during vision perception by the retinal cones and rods.

3. Maintain the proper physiological milieu for the photoreceptors and their organelle to function; at the same time arrest any evolving pathological conditions.

The body is made up of many diverse types of cells composed of different types of molecules. Molecules are made up of one or more atoms bound to each other forming a molecule—i.e. one or more elements joined by chemical bonds. The atoms consist of a nucleus, a mix of postively charged protons, electrically neutral neutrons, and the central nucleus surrounded by a cloud of negtively charged electrons bound to nuclues by electromagnetic force. The number of protons (positively charged particles) in the atom's nucleus determines the number of electrons (negatively charged particles) surrounding the atom.

Electrons are involved in chemical reactions which the electrons are the substance that bonds atoms together to form molecules. Electrons surround, or “orbit” an atom in one or more shells. The innermost shell is full when it has two electrons. When the first shell is full, electrons fill the second shell. When the second shell has eight electrons, the shell is full, and the process continues. Free radicals are oxygen atoms. The oxygen atoms are missing one electron from the pair which the atoms are endowed naturally. When an atom is missing an electron from a pair, the atom becomes unstable and reactive which the atom wants to find another electron (ROS) to fill in the missing electron in the gap. Hence, the atom grabs an electron from the next atom. When the atom is near, a free radical seizes an electron from another atom, the second atom becomes a free radical which this process starts a cascade of new free radicals in our body like the atomic chain reaction. Once the process is started, the process can continue which the process results in the disruption of a living cell function leading to disease states of many kinds from retinitis pigmentosa to cancers.

There are numerous types of free radicals formed within the body. We focus on the oxygen-centered free radicals or ROS; because the retina and the photoreceptors are very sensitive to oxygen which the effect is free radicals. The majority of common ROS incorporate: 1. the superoxide anion (O2-), 2. the hydroxyl radical (OH.), 3. singlet oxygen (1O2), and 3. hydrogen peroxide (H2O2) Superoxide anions are formed when oxygen (O2) acquires an additional electron, which the molecule is the only one unpaired electron. For example, the hydrogen peroxide is produced where the H₂O₂ can be converted to the highly damaging hydroxyl radical or be catalyzed or excreted harmlessly as water. Glutathione peroxidase is essential for the conversion of glutathione to oxidized glutathione which H₂O₂ is converted to water. If H₂O₂ is not converted into water, one O₂, singlet oxygen, is formed which is not a free radical. The singlet oxygen can act as a catalyst for the free radical formation. The molecule can interact with other molecules leading to the formation of a new free radical. Zinc is one of the most important metals, which zinc exists in one valence (Zn2+) which the Zinc does not catalyze free radical formation.

Retinitis pigmentosa results due to damage by ROS in a genetically defective rods, besides other etiological factors. This is substantiated by delay in progression of the disease by the use of Vtiamin A, E, and C which these vitamins are important known antioxidants. Zinc, unlike, other metals acts to stop free radical formation by displacing those metals which the metals do have more than one valence including iron. Every time the light comes in contact with the photoreceptors, the mitochondria O2- is endlessly being formed. Our invention of using insulin and other therapeutic agents reduces these ROS, prevent the photoreceptors damage, and augment the protection of the photoreceptors, which this process prevents further damage where the progression of retinitis pigmentosa is delayed or halted.

What do the free radcials do once they are formed? The free radicals stagger, stumble, splash around, and seize electrons from adjacent cells-which the free radicals do an assortment of damage to them at the same time. The ultraviolet light in sunshine (skin cancer and cataracts); Toxins of all sort, such as: tobacco smoke; the chemicals found in our food with lack of antioxidants; the poisonous wastes of our bodies own metabolism; and man-made toxins like air pollution, drugs, and pesticides are some of the culprits. On an average, every cell in our body comes under attack from free radicals once every ten seconds which the cell attack is blamed for cancers, heart diseases, retinitis pigmentosa, neurodegenerative diseases, and a host of other diseases.

Sometimes the body's immune systems' cells purposefully create free radicals to neutralize viruses and bacteria. The photosensitive cells of the retina in essence avacular are easily subject to free radicle damage due to light hitting the receptors continuously for amost 16 hours a day. If the photoreceptors are genetically defective, the production of ROS, and the effect of ROS is amplified where the results are in their dysfunction and damage, ultimately, apoptosis contributes to the retinitis pigmentosa of the eyes with segmental or total loss of vision. The light from the sun or other sources will generate free radicals which the radicals can cause more damage. The free radicals accelarate the retinitis pigmentosa development that RP leads to blindness if there is no innate (inherent) defense against ROS. In retinitis pigmentosa the defense against ROS is inhibited, lacking, or missing. Our invention of use of insulin with antioxidants such as Vitamin A, E, C, GLA, Omega 3, and Glutathione and other natural supplements can be of immense therapeutic value in treating this condition.

Normally, the body can't handle free radicals if antioxidants are unavailable, or if the free-radical production becomes excessive as seen in retinitis pigmentosa due to constant bombardment of light on the photoreceptors. The results will be damage to the retina. Free radicals are present in all living cells. Free radicals are a part of the cell metabolic life processes. Free radicals have a incredibly short half-life, hence, the free radicals are not easy to measure in the laboratory which the short half life of the free radical increases the expense to study and to test. However, excessive free radicals in our cells can attack the cell membranes (the outer coat of the cell and delicate folded lamellae of rods and cones outer segments) where the free radicals cause the cell and the tissue damage. Free radicals, besides attack on cell membranes (bilamillar lipid protein complex), intracelluar organelle, they can also break strands of DNA (the genetic material in the cell nucleus).

The broken strands of DNA where the chemicals proved to cause cancer by forming free radicals. From the above description, the obvious is where the ROS generated due to the light perception. The ROS associated metabolic processes play an important role in retinitis pigmentosa. Our invention of insulin will help to curtail ROS. This is similar to the insulin protective effects on the myocardium of the heart in the cardioplegic solutions after open heart surgery, heart attack, and drive the potassium inside or dirve out of the cells using GIK infusion as discussed above.

Experimental studies show that the cone and rod photoreceptors remaining in many retinitis pigmentosa patients functions normally for their numbers with the amounts remaining visual pigment which the belief support an idea that these photoreceptors can be rescued (Eliot L. Berson. Retinitis Pigmentosa. The Friedenwald Lecture Investigative Opthalmology and Visual Science, April 1993, Vol. 34, No. 5, 1659-1676). Our invention using insulin ophthalmic preparations with nurticeuticals and other therapeutic agents can rescue these remaining photoreceptors, prevent their progression to apoptosis, maintain the remaining vision preceived by these photoreceptors and prevent the progression of retinitis pigmentosa which RP can lead to total blindness. Risk or hazard factor investigation analysis of well-defined populations studied over time may reveal ameliorating or aggravating factors associated with the course of the disease. The possible implications for prophalactic therapies used in our invention described herein.

### The Following are Examples of Using Our Invention of Insulin and/or IGF-1 Biological Factors Alone or in Combination With known therapeutic, pharmaceutical, biochemical, Nutracuetical, and Biological Agents or Compounds to Treat Retinitis pigmentosa and other associated oculopathies

### Example 1

Select the patient establish the type of retinitis pigmentosa and the RP etiology which the person is suffering. The complete examination of the eye as described above is important, Record the preliminary examination results on the patient chart. The patient will be examined for any corneal, conjunctival, and retinal BV afflictions by using marker dyes. Position the patient in a supine posture or sitting with the head hyper extened with a support. Using a dropper or dropper bottle containing the insulin formulations are instilled two or three drops of insulin preparation in each eye lower lid formix and/or everted upper eyelid. Both eyes receive the eye drops. Apply slight pressure at the nasal angle of eye on the nasolacrimal canaliculi-sac-duct system to prevent leaking of the therapeutic agents to the nose to avoid systemic absorption. The adverse effects can be prevented or minimized using the method shown in the FIG. 6.

The patient must remain stationary for 2-3-5 minutes. The patient can resume the desired posture after the patient has been stationary for 2 to 5 minutes. These instructions should be given to the patients. The patient or the caregiver should be trained to apply the ophthalmic drops using sterile methods for the treatment of retinitis pigmentosa with our inventive eye drops which the eye drops contain insulin, appropriately. The insulin ophthalmic therapeutic drops are used before going to bed and after getting up from bed in the morning, after taking a shower as well as before taking a nap in the afternoon.

### Example 2

Follow the instruction as described in the above EXAMPLE 1.

If the retinitis pigmentosa is associated with keratoconus sicca, use a topical FDA approved emulsion of cyclosporin for treating the associated condition (Restasis™, Allergan, Inc., and Irvine, Calif.). The emulsion is a mixture of cyclosporin combined with a higher fatty acid glyceride, such as castor oil, and a surface active agent, such as polysorbate 80, and an emulsion stabilizer, such as a cross-linked polyacrylate. This acts by decreasing the inflammation on the eye surface (probably eye lid tear glandular system). The emulsion helps to increase the production of healthy tears. However, treatment with an emulsion containing oily droplets can result in eye irritation or a clouding of visual field. The emulsion may delay the absorption of insulin.

The oily consistency of this preparation makes the active ingredient less bioavailable. Restasis is not appropriate for immediate relief for an uncomfortable irritated eye as the results may take up to 6 months for maximum improvement (source: The Eye Digest). The addition of insulin will make the preparation more effective which the Insulin can enhance the uptake of cyclosporin, and augment/amplify the effects of the cyclosporins in the preparation. This biological effect requires less cyclosporine which insulin can be added in the final cyclosporin preparation; at the same time. There will be a decrease of time needed inside the afflicted cells to achieve the desired effects. The use of insulin before or with the preparation will enhance the activity of Restasis. The insulin will cause the Restatasis to become more effective within days instead of months due to augmentation/amplification effects of insulin. We prefer to use water soluble solution of cyclosporin as described. Then apply one drop of aqueous cyclosporin in water soluble eye preparation as formulated in the invention. Insulin can enhance the uptake of water soluble cyclosporin more efficiently than oil soluble preparations; augment and amplify the effects of the cyclosporins on the structures involved in development of retinitis pigmentosa associated with this oculopathies.

### Example 3

Follow the instruction as described in the above EXAMPLE 1. If the men and woman suffer from retinitis pigmentosa with dry eyes syndrome due to estrogen and tesosterone deficiency. They can be treated with estorgen and testosterone opthalmic drops with insulin. Androgens are believed to be trophic factors for various glandualar and neuronal tissues including the retina. The androgens exert potent anti-inflammatory activity through the production of transforming growth factor beta (TGF-beta), suppressing lymphocytic infiltration, inflammatory response in the pigment epithelium, and the retina and the associated blood vessels.

The eye drops containing testosterone can be prepared which the drops can be used after pretreatment with insulin. The ophthalmic drops can be prepared using testosterone (androgen), DHEA—a mild androgen, cyclosporin. Insulin can be used to treat retinitis pigmentosa with the dry eyes syndrome, Sjogren's syndrome, and KCS at the same time. Our preliminary studies indicate, that the preparation for these syndromes are easy to prepare. These ophthalmic eye preparations with insulin are used to treat retinitis pigmentosa associated with these oculopathies.

### Example 4

Follow the instruction as described in the above EXAMPLE 1. Previous, investigations demonstrated that bendazac prevents protein denaturation produced by U.V. rays. The bendazac is capable of attenuating the biological effects of sun radiations and the tissue associated with ROS on the retina. This possibility was confirmed by the recent observation that bendazac has a protective effect on photo-oxidative processes linked to free radicals involved in the retinitis pigmentosa. The photosensitizing effect seemingly linked to the formation of free radicals (ROS) as described above where free radicals damages the photoreceptors.

The ophthalmic solution of 1% lysine salt of bendazac can be used with insulin. Our invention enhances therapeutic agents to reach the site of pathology in the retina. Lysine salt of bendazac at the oral dose of 500 mgs/three times daily for a period of 6 months are administered when using insulin and bendazac ophthalmic preparations to augment the therapeutic agents effect.

### Example 5

Follow the instruction as described in the above EXAMPLE 1. Then use the pharmaceutical kit for treatment of retinitis pigmentosa containing the enzymes glutathione peroxidase (Enzyme A), prolidase (Enzyme B), glucose-6-phosphate dehydrogenase (Enzyme C), optionally, aldose reductase (Enzyme D) in aliquot parts and interactive quantities appropriate, for administering ophthalmic drops for approximately three consecutive days, at monthly intervals, for about three months for each eye as disclosed U.S. Patent Application Publication Number: 2006/0134088 A1. These therapeutic agents are used in combination with insulin before, during, or after application of these ophthalmic drops.

### Example 6

Follow the instruction as described in the above EXAMPLE 1. U.S. Pat. No. 7,037,943 B2 discloses a method for treating or preventing retinal pathology or injury by placing a retinal stimulating substance in the eye between the internal limiting membrane and the retina, which the internal limiting membrane is the target site for the substance. The substance may be an implant that provides electrical stimulation to adjacent ganglion and neurofiber cells.

Alternatively, the substance may be a pharmaceutical substance to stimulate the retina. In addition to providing direct contact, the substance has its target. The method obviates the need for artificial structures which the structures are tacks or adhesives which the artificial structures may cause retinal bleeding or traction. Our invention of using insulin ophthalmic drops with semi surgical therapeutic procedure helps to contain the disease of retinitis pigmentosa much more effectively.

### Example 7

Follow the instruction as described in the above EXAMPLE 1. U.S. Pat. No. 5,948,801 discloses the use of Brinzolamide as eye drops, systemically between 250 to 1000 mg orally, or intravitreal up to 10 mg per eye or periocular up to 50 mg per eye to treat retinal edema and the condition associated with retinitis pigmentosa. We incorporate Brinzolamide ophthalmic drops to treat oculopathies of various kinds including retinitis pigmentosa combined with insulin ophthalmic drops to maintain the integrity of RPE cell layer by decreasing the edema where the relief of the edema can play a role in alleviating the condition of retinitis pigmentosa.

### Example 8

Follow the instruction as described in the above EXAMPLE 1. U.S. Pat. No. 6,716,835 B1 discloses a method of retarding degeneration of retinal photoreceptors comprising administeration to a patient afflicted with age-related macular degeneration or retinitis pigmentosa. A therapeutically effective amount of a compound selected from the group consisting of calcium channel blocker compounds and/or cyclic GMP-dependent channels, namely diltiazem, for treating retinal pathologies, and more particularly retinal diseases caused by degeneration of visual receptors. The diltiazem can be formulated as an ophthalmic preparation with insulin to be used and to treat retinitis pigmentosa in our invention.

### Example 9

Follow the instruction as described in the above EXAMPLE 1. U.S. Patent Application Publication Number: 2001/0049369 A1 demonstrates that brimonidine tartrate, a potent alpha-2 adrenergic receptor agonist, applied topically to the eyes can prevent photoreceptor cell degeneration. The Muller cell associated with degenerative signs in an in vitro model of retinal degeneration and retinal detachment. Brimonidine allowed for the formation of highly structured photoreceptor outer segments, prevented the expression of stress markers in Muller cells, and preserved the expression patterns of Muller cell markers of proper cell to cell contact and differentiation.

Ultra structural studies indicated that Brimonidine favored the formation of cell to cell junctions between photoreceptor cells. The Muller cells with the cell to cell junctions indicate that this phenomenon is associated with the exertion of the neuroprotective effect. The results suggests that brimonidine compounds may be utilized as an effective therapeutic agent for early and late onset retinal degenerations caused by defects in photoreceptor cells, Muller cells or both, as an adjuvant to therapeutic success in retinal detachment surgery or macular translocation surgery for age-related macular degeneration and retinitis pigmentosa. This therapeutic agent has been used for treatment of chronic open angle glaucoma, also. Our inventive method uses brimonidine with insulin ophthalmic drops to enhance its uptake for augmentation/amplification effects on the photoreceptors cells, and other components of retina to prevent oculopathies including retinitis pigmentosa.

### Example 10

Follow the instruction as described in the above EXAMPLE 1. U.S. Patent Application Publication Number: 2009/0060980 A1 discloses a novel method of treatment for retinal diseases and conditions including age-related macular degeneration, genetic-based retinal degenerations, and retinal detachment. A novel glycan binding protein is thought to be a cell surface receptor that the cell has been discovered in the retina. The retinal glycan binding receptor is shown to play an important role in promoting assembly of outer segment (OS) membranes by the photoreceptor cells of the eye, a process that is essential for vision.

Based on the finding, certain sugars can bind with very high affinity to the retinal glycan receptor which the sugars stimulate the retinal glycan function. The invention provides novel therapeutic agents for treatment of retinal diseases that are multivalent N-linked glycans. Preferred pharmaceutical compositions in accordance with the present invention comprise active agents having the general formula: (Gal-GlcNAc)-Man3₃-GlcNAc_{z}, where n is 1-4. Particularly preferred multivalent glycans are galactosylated, biantennary (NA2), also, galactosylated, triantennary (NA3) oligosaccharides. We want to incorporate insulin which our invention can be used with these oligosaccharides to treat retinitis pigmentosa and other oculopathies such as age related macular degeneration, and retinal detachment.

### Example 11

Follow the instruction as described in the above EXAMPLE 1. The presently disclosed U.S. Patent Application Publication Number: 2009/0053816 A1 provides methods of diagnosing retinal disorders in subjects by measuring hemoglobin and measuring modified hemoglobin in the subjects. The presently disclosed subject matter provides methods of treating retinal disorders in subjects by decreasing hypoxia in retinal tissue of the subjects through modulation of hemoglobin levels and activities in the retinal tissue. Our inventive method uses insulin ophthalmic instillation to the method of modulating hemoglobin as described in the above patent, which the insulin will enhance the activity where the insulin will reduce the likelihood of hypoxic damage of photoreceptors which the hypoxic damage leads to retinitis pigmentosa development or aggravates the existing disease.

### Example 12

Follow the instruction as described in the above EXAMPLE 1. Antibodies are proteins that the antibodies are generated by the immune system's white blood cells. The antibodies circulate in the blood which the antibodies attach to foreign proteins called antigens in order to destroy or to neutralize them which the antibodies help rid the systemic infection or eliminate foreign proteins harmful to the body cells. Monoclonal antibodies are laboratory created or fashioned substances that the antibodies can locate. The antibodies bind to specific molecules such as tumor necrosis factor (TNF) which the TNF is a protein involved in causing the inflammation and the damage of autoimmune diseases. There are many MAB such as: Remicade™, Etanercept, Embrel™, and Humira™. The TNF and anti TNF agents are on the market to treat autoimmune bodies.

Etanercept is a drug that the drug is used to treat autoimmune diseases by interfering with the tumor necrosis factor (TNF, a part of the immune system) by acting as a TNF inhibitor. This is given 25-50 mg. Humira administered by injection is produced from human proteins. The newest monoclonal protein to be approved for the treatment of rheumatoid arthritis is Rituxan. Infliximab (Remicade) is a chimeric mouse/human monoclinal antibody given by intravenous infusion the monoclonal protein works by binding to tumor necrosis factor alpha (TNFα). Several new monoclonal antibodies are in the development stage to treat autoimmune diseases.

Multiple monoclonal antibodies are currently under investigation for the treatment of retinitis pigmentosa (Meijer jM, Pijpe j, Bootsma H, Vissink A, Kallenberg C G (lune 2007). “The future of biologic agents in the treatment of “Sjögren's syndrome”. Clin Rev Allergy Immunol 32 (3): 292-7). All TNF inhibitors are immunosuppressants. We formulate Etanercept (Embrel) using no more than 200 μg per ml of ophthalmic solution which these results in 10 μg per drop instilled. The final solution will have insulin as described above to reduce the nonspecific inflammatory processes in the photoreceptors in retinitis pigmentosa caused by ROS. The patient should use the insulin and MAB preparations once or twice a day. The dose of MAB used in our invention is minuscule; to take into account any contraindications with tuberculosis or tumors while using these biological therapeutic agents with our invention insulin.

### Example 13

Follow the instruction as described in the above EXAMPLE 1. The hyaluronic acid (HA) is produced by fermenting the bacterial strain Bacillus subtilis. It is the world's first pure HA that is 100% free of animal-derived raw materials and organic-solvent remnants. Hyaluronic acid is a novel viscosity enhancer for use in topical eye care formulations which hyaluronic acid is filterable. The hyaluronic acid is heat stabile with pH (0.1% solution) 6.0-7.5 which this is desired to treat retinitis pigmentosa and other oculopathies. The HA can be a key ingredient for topical ophthalmic formulations. The hyaluronic acid is a natural compound which the compound is biocompatible, non-immunogenic, and biodegradable.

This compound is one of the most hygroscopic molecules found in nature; hydrated hyaluronic acid can contain up to 1.000-fold more water than its own weight. These exceptional water retention properties result in enhanced hydration of the corneal surface; retain the active therapeutic agents to be slowly released to be absorbed and transported to the site of retinitis pigmentosa. Moreover, applications of ophthalmic formulations containing HA reduce tear elimination which HA enhances pre corneal tear film stability. The HA has a useful property against retinitis pigmentosa. The muco-adhesivity of hyaluronic acid provides effective coating and long lasting protection of the cornea and conjunctival sac due to the extended stay, water retention quality, and accommodation times on the ocular surface. When topically instilled on the eye with insulin, HA promotes physiological wound healing by stimulating corneal epithelial migration and proliferation of keratocytes.

HA enhances the healing of photoreceptors which HA acts as therapeutic agents for treatment of retinitis pigmentosa with other oculopathies. HA has the viscosity-enhancing agent of choice, decreases the drainage rate of ophthalmic solutions where the HA allows the insulin to be absorbed into deep eye structures including the retina. Our invention of using insulin before and after the application of the HA with or without other anti retinitis pigmentosa therapeutic agents combining with insulin in the final formulation can effectively prevent, curtail, and cure the retinitis pigmentosa associated with or without other oculopathies.

### Example 14

Follow the instruction as described in the above EXAMPLE 1. Mitoxantrone (Novantrone) is a chemotherapeutic drug that the drug works by suppressing the immune system. Mitoxantrone is used to slow the worsening of neurologic disability and to reduce the relapse rate in patients with clinically worsening forms of relapsing-remitting and secondary progressive MS. Mitoxantrone is a DNA-reactive agent, that agent intercalates into deoxyribonucleic acid (DNA) through hydrogen bonding, where the Mitoxantrone causes crosslink's and strand breaks. Mitoxantrone interferes with ribonucleic acid (RNA). Mitoxantrone is a potent inhibitor of topoisomerase II, an enzyme responsible for uncoiling and for repairing damaged DNA especially in photoreceptors cells of retinitis pigmentosa. Mitoxantrone can be prepared in doses of 100 μg/ml by premixing with insulin. These drops can be effective in autoimmune related retinitis pigmentosa.

### Example 15

Follow the instruction as described in the above EXAMPLE 1. Corticosteroids are the most commonly used treatment for autoimmune diseases, allergic conditions, insect bites, septic shock, and many other conditions including retinitis pigmentosa. The corticosteroids are given to reduce the inflammation. Examples included are oral prednisone and intravenous methyl prednisolone. Lotemax, an ophthalmic corticosteroid, targets inflammation with a unique site-active mechanism of action.

Structural modifications associated with an ester ophthalmic steroid, which Lotemax make highly lipid soluble, enhancing the penetration into cells and enabling Lotemax to exert anti-inflammatory activity within the eye. Pre-treating with insulin or combining with insulin ophthalmic drops can enhance the uptake of these corticosteroids and relive retinitis pigmentosa and other autoimmune afflictions of the eye. The insulin with steroid attenuates the effects of ROS mediated photoreceptor cells damage, stabilizes the membranes of the photoreceptors, and their organelle which restores function and health.

### Example 16

Follow the instruction as described in the above EXAMPLE 1. Studies on experimental animals retinal pigment epithelium (RPE) showed, that RPE actively secretes sodium and calcium into the retinal space, which the space absorbs chlorine and maybe bicarbonate and potassium. This activity could be important in controlling the ionic milieu in the outer retina. (Miller, et al., “Active Transport of Ions Across Frog Retinal Pigment Epithelium,” Experimental Eye Research, 25:235-248 (1977)). Acetazolamide have been used in glaucoma and has application in preventing or slowing the spread of retinal detachments or hastens reabsorption of subretinal fluid if retinitis pigmentosa is associated with uveal and macular edema. U.S. Pat. No. 5,948,801 discloses Methods for preventing and treating retinal edema with brinzolamide similar to Acetazolamide are disclosed. It has also been shown to be effective in the treatment of chronic macular edema associated with retinitis pigmentosa (Gerald A. Fishman, M D; Leonardo D. Gilbert, C O T; Richard G. Fiscella, R Ph, M P H; Alan E. Kimura, M D; Lee M. Jampol, M D. Acetazolamide for Treatment of Chronic Macular Edema in Retinitis Pigmentosa. Arch Opthalmol. 1989; 107(10):1445-1452).

Acetazolamide is more effective improving the macular edema compared to brindorzolamde. Photoreceptors dysfunctioned in a roundabout way. Retinitis Pigmentosa may be related to retinal piment epithelium edema resulting in disruption of photoreceptors function. In our invention we want to use ophthalmic drops containing Brinzolamide, and/or Acetazolamide with insulin in retinitis pigmentosa to relive swelling of the pigment epithelium which the insulin would restore the function to maintain the photoreceptors cells.

### Example 17

Follow the instruction as described in the above EXAMPLE 1. There are two types of fatty acids needed for health which these fatty acids are used by millions every day as health nurticeuticals supplement. One is Omega 3 and the other is Omega 6. Omega 3 fatty acids include: Alpha-linolenic acid (ALA), Eicosapentaenoic acid (EPA), Docosahexaenoic acid (DHA); and the Omega 6 fatty acids include: Linoleic acid (LA), Gamma linolenic acid (GLA), Dihomo-gamma-linolenic acid (DGLA) and Arachidonic acid (AA). Gamma-linolenic acid (GLA) is an omega-6 fatty acid found mostly in plant-based oils. GLA is considered an essential fatty acids and antioxidants. These fatty acids need to be supplemented. They are necessary for human health but the body isn't capable of producing the fatty acids. Hence, the fatty acids have to be obtained through every day food. Omega-6 fatty acids with omega-3 fatty acids, also, known as polyunsaturated fatty acids (PUFAs). These play a vital role in brain function, its normal growth and development, which the retina is part. They help to stimulate skin, hair growth, maintain bone health, regulate metabolism, and maintain the reproductive system. To maintain health, the ratio of omega-6 to omega-3 fatty acids consumed should be the ratios of 10:1 to 5:1; previously, it was 15:1.

The latest studies show that the approximately 8% of the brain's weight is comprised of omega-3 fatty acids (DHA and EPA) (O'Brien J S, Sampson E L. Lipid composition of the normal human brain: gray matter, white matter, and myelin. J Lipid Res. 1965 October; 6(4):537.44), the building block for an estimated 100 billion neurons (Chang C Y, Ke D S, Chen J Y Essential fatty acids and human brain. Acta Neurol Taiwan. 2009 December; IS(4):231-241). Omega 3 fish oil contains two active ingredients: EPA (Eicosapentaenoic Acid) and DHA (Docosahexaenoic Acid). They are interconvertible in the brain. They play a host of vital roles in neuronal structure and function, protecting the neural structure from oxidative damage, inflammation, and the cumulative destruction inflicted by other chronic insults.

The retina is an extension of the brain with millions of photoreceptors and other neurons. The Omega 3 fatty acids can protect the photoreceptors from oxidative damage, inflammation, and the cumulative destruction inflicted by other chronic insults as they do with CNS. Embedded in the omega-3 DHA-rich retinal photoreceptors and neuronal membranes are numerous proteins with complex molecules required for electrochemical transmission, signal reception, and transduction. Scientists have recently shown that the precise balance of fatty acids in brain cells helps to determine whether a given nerve cell in the retina will be protected against injury or inflammation, or whether it will succumb to the injury (Julius Goepp. Omega 3 Fatty Acids increase Brain Volume while reversing many aspects of neurologic aging. Life Extension, August 2010, Pages 56-61).

A remarkable animal study has revealed that omega-3 fatty acids halt the age-related loss of brain cell receptors vital to memory production which the fatty acids show potential for increasing neuronal growth (Dyall S C, Michael G J, Michael-Titus A T. Omega-3 fatty acids reverse age-related and Omega-3 fatty acids decreases in nuclear receptors and increase neurogenesis in old rats. J Neurosci Res. 20 I 0 March 24). Animal studies suggest that oral supplementation with DHA may enhance the formation of new synapses and their vital dendritic spines. The supplementation can improve cognitive function (Wurtman R1, Cansev M, Ulus R H. Synapse formation is enhanced by oral administration of uridine and DHA, the circulating precursors of brain phosphatides. J Nutr Health Aging 2009 March; 13(3): 189-97).

Again, the retina being part of the brain and the brains' extension, DHA, and EPA will have the same effect on the photoreceptors and other neurons of the retina. These fatty acids can improve their synapses function; prevent damage to the vision caused by retinitis pigmentosa, and other oculopathies. Omega 3 significantly reduced levels of inflammatory cytokines circulating in the blood. This suggests that the brain and retinal tissue inflammation can be alleviated or tone down in retinitis pigmentosa. The molecular basis for this early intervention strategy lies in the photoreceptors cellular pathophysiology at the core of the retinitis pigmentosa: omega-3 treatment of cultured brain cells suppresses many of the early signs of damage triggered by the inflammatory protein known which this includes the beta amyloidal of Alzheimer's (Ma Q L, Yang F, Rosario E R, et al. Beta-amyloid oligomers induces phosphorylation of tau and inactivation of insulin receptor substrate via c-lun N-terminal kinase signaling: suppression by omega-3 fatty acids and curcumin. j Neurasci. 2009. 15; 29 (28):9078-89).

Most omega-6 fatty acids in our diet come from vegetable oils in the form of linoleic acid (LA). Salmon and related fish are a rich source of omega complexes EPA and DPA (Docosapentaenoic acid). 33% of the long chain Omega-3 fatty acids circulating in human blood is attributable to DPA. The BV wall can convert EPA to DPA as the effective agent. The body converts linoleic acid to GLA and then to arachidonic acid (AA). GLA can be obtained from several plant-based oils including evening primrose oil, borage oil, and black currant seed oil. A healthy diet should contains a balance of omega-3 and omega-6 fatty acids. The Omega-3 fatty acids help to reduce inflammation in photoreceptors.

Our invention of using insulin ophthalmic drops with Omega 3 fatty acids can be applied to the eyes. They can be prepared with mixing of Vitamin A. The patient takes orally DHA 1,700 mg combined with 600 mg EPA Omega 3 fatty acid (DPA-EPA). The patient should wait 30 to 60 minutes for the DHA-EPA to be absorbed and to reach high plasma levels. Then insulin drops should be applied to the eyes one hour later which the insulin will enhance the uptake of omega 3 from the choriocapillares by photoreceptors. The insulin will make the omega 3 more effective in the treatment of retinitis pigmentosa and other oculopathies. Insulin and Omega 3 ophthalmic drops can be formulated to treat RP.

### Example 18

Follow the instruction as described in the above EXAMPLE 1. There is high incidence keratoconjunctivitis sicca in postmenopausal women with symptoms ranging from mild foreign body, pain and even visual loss due to ocular surface abnormalities including retinitis pigmentosa. The use of conjugated estrogens decades ago to treat KCS was indicated (Bohigian, G. Handbook of External Diseases of the Eye (Alcon, Inc.) 1980, p. 79). U.S. Pat. No. 5,041,434; U.S. Pat. No. Re. 34,578; and U.S. Pat. No. 6,096,733 describe the use of estrogens. The latter patent disclosed very small doses of 17-β-estradiol Compounded with polysorbate 80 (USP), povidone (USP) (K-30 type), hydroxyethylcellulose (USP), sodium chloride (USP), disodium EDTA (USP), benzalkonium chloride (USP), dilute HCL for pH adjustment and purified water (USP) qs. As described in our invention pre-treating the affected eyes with insulin or adding to the above preparation of estradiol eye drop can enhance the local therapeutic effect by insulin mediated augmentation-amplification effects. This invention will provide the needed relief much faster without systemic effect if the condition is associated with retinitis pigmentosa.

### Example 19

Follow the instruction as described in the above EXAMPLE 1. The symptoms of an eye allergy are mild to moderate where allergies can be severe during early spring and the beginning of fall. Self treatment to avoid allergens, to irrigate the eyes with saline (salt solution) and to place the ice packs, and the cold water compresses on eyes which this may not be effective in a severe case. The medical treatment is needed to relieve them. Retinitis pigmentosa associated with severe allergic conjunctivitis. This condition isn't helped by other treatments. Conjunctivitis may benefit from specific allergen immunotherapy (desensitization) which the therapy is usually effective. Most commonly used and prescribed medications are: levocabastine (brand name Livostin); antihistamines (antolozine) together with a medicine that constricts blood vessels (naphazoline, phenylephrine); sodium cromoglycate (4%); non-steroidal anti-inflammatory (NSAID) eye drops; and steroids (hydrocortisone, Dexamethasone, prednisolone). Eye drops containing anti allergic, vasoconstrictors, and cortisone, can be used long term to treat retinitis pigmentosa and allergic conditions. The drops with insulin applied before the use of the above described therapeutic agents.

Our experimental data using insulin with vasoconstrictors and anti allergic therapeutic agents such as corticosteroids supports that the allergic condition is relieved rapidly; the red eye disappeared with prolonged effect when insulin was added to the ophthalmic therapeutic agents which the insulin can adversely affect the retinitis pigmentosa.

### Example 20

Follow the instruction as described in the above EXAMPLE 1. Testosterone has trophic effect on the neurological structures. Studies has shown subjectively the patients felt better when DHEA ophthalmic drops were use compared to the artificial tears or testosterone as artificial tears (Connor C G, and Fender J. Comparison of Androgenic Supplemented Artificial Tears. Invest Opthalmol V is Sci 2002; 43: E-Abstract 66; Schaumberg D A, Sullivan DA, Dana M R. Epidemiology of retinitis pigmentosa. Adv Exper Med Biol 2002; 506: 989-998. Schaumberg D A, Sullivan D A, Buring J E, Dana M R. Prevalence of retinitis pigmentosa among US women. Am J Ophth 2003; 136:318-326). These study supports the previous studies by Notion and Sullivan that addition of androgenic hormones to artificial tears benefit various oculopathies. DHEA is known as dehydroepiandrosterone.

This is a steroid hormone produced by the adrenal glands where the DHEA is converted to other hormones like estrogen and testosterone. DHEA is a steroid hormone produced naturally by the adrenal glands that has 5% of the androgenic activity of testosterone. Our invention relates the use of testosterone or DHEA eye drops with insulin. Use the insulin drops before the application of the androgenic eye preparation. These hormonal eye drops in combination with insulin can also be prepared and used as ophthalmic drops to treat these conditions associated with retinitis pigmentosa.

### Example 21

Follow the instruction as described in the above EXAMPLE 1. A method of topically instilling insulin drops to a person or animals conjunctival sac to treat retinitis pigmentosa with administration of insulin. The insulin enhances their uptake. The insulin has therapeutic activity by entering into afflicted structures in the eye. This can be combined with uptake facilitators such electroporation, iontophoresis, sonophoresis, vibroacoustic, vibration, and other physical (heat, magnetic force, radio frequency, microwave, laser lights etc.) methods with other appropriate therapeutic, biological, pharmacological anti-glaucoma, and retinal protectors. These agents combined with insulin therapy as described. These methods can be used as prophylaxis, to diagnose, prevent and to treat the above conditions.

### Example 22

Follow the instruction as described in the above EXAMPLE 1. Deferoxamine is a chelating agent used to remove excess iron from the body. Iron removed which the reduction reduces the damage done to various organs and tissues, such as the liver, CNS, and retina. The damage that we saw in the retina can be due to excessive iron from the choroid and retinal blood vessels leaking excessive iron reacting with ROS where the excess damages the sensitive photoreceptors. The role of iron (metallobiology) in neurodegenerative disorders has long been implicated with particular attention given to iron.

Iron is one of the most important redox metals which iron has been largely linked to senile toxicity and neurodegenerative disorders which the disorders are as follows: Alzheimer's, MS, and Parkinson's diseases and aging patients (Stankiewicz J M, Brass S D (2009) Role of iron in neurotoxicity: a cause for concern in the elderly? Curr Opin Clin Nutr Metab Care 12:22-9). The redox switching capability of iron from ferrous to ferric state, and vice versa, makes iron one of the most dangerous catalytic elements responsible for the retinal and other neurodegenerative process resulting in diseases and dysfunction. Iron generates free radicals where the free radicals are reactive with the oxygen species in the aged tissue as evidenced by higher heme oxygenase-I, which this contributes to increased susceptibility, to oxidative stress with aging (Hirose W, Ikematsu K, Tsuda R (2003) Age-associated increase in heme oxygenase-1 and ferritin immunoreactivity in the autopsied brain. Leg Med 5(Suppl.1):360-6).

The nerve tissue of the photoreceptors are exposed to the iron will not spare from the iron effects of neurodegenerative process. Biochemical events surrounding iron-mediated catalytic events which the biochemical events give rise to oxidative stress and free radical generation that the events damages photoreceptors in retinitis pigmentosa. The damage is described and the damage is known as the Fenton reaction as indicated below:

Fe3⁺.O²→KO2-Fe2⁺+O₂ (Step I);

Fe²++H₂O₂—Fe³⁺+OH—+KOH (Step II)

O2⁻+H₂O₂→.HO⁻+O2 Combining Step I and II

The role of iron in the neurodegenerative process which the retina is part of the nervous system can be best described in three distinct phases: 1. accumulation in choroidal blood vessel walls and Bruch's membrane, 2. invasion through the RPE from the Bruch's membrane, and 3. catalytic activity against the outer segment of the photoreceptors. A recent study shows that iron chelation can speed the healing of nerve damage in retinitis pigmentosa where iron chelation can reduced or curtailed RP. The use of deferoxamine as iron chelator with our invention insulin can have dramatic curing and/or curtailing effect on the MS, Alzheimer's, Parkinson's, ALS, dementia with Lewy bodies, and other degenerative diseases of the CNS including senile brain atrophy.

Deferoxamine may modulate expression and release of inflammatory mediators in the retinitis pigmentosa as indicated in Fenton reaction by specific cell types, thus, reduce or stop the damage by our invention. Deferoxamine used with insulin of our invention along with ophthalmic drops can reduce the ROS oxidant damage, arrest, or delay the processes of retinitis pigmentosa. We have used this method to treat the CNS disease with good results. We have used the extract of Turmeric, called curcumin, with insulin as antioxidant with good results. Curcumin is safe and isn't toxic to the retina or the CNS.

### Example 23

Follow the instruction as described in the above EXAMPLE 1. Another drug available to treat autoimmune disease related to Sjogren's disease is an organo sulfur compound, anethole dithiolethione (ADT-trade name Sialor, sold over the counter in Canada) with hardly any side effects. The ADT stimulates the secretion of saliva, in patients with autoimmune exocrinopathy (Sjogren's syndrome). Sialor alleviates the symptoms of xerostomia and xeroophthalmia. We have used ADT 25 mg orally and ADT in nanograms concentration in liquid ophthalmic eye drops with successes in these conditions, especially, those on chemotherapy, menopausal women, and chronic smokers with dry mouth and dry eyes conditions.

There is secretory dysfunction associated with RPE and Muller cells which are needed for proper functioning of the photoreceptors by removing ROS. This can be one of the important non toxic oral and eye drops for the treatment of retinitis pigmentosa (Ben-Mandi M H, Gozin A, Driss F, Andrieu V, Christen M O, Pasquier C. Anethole dithiol ethione regulates oxidant-induced tyrosine kinase activation in endothelial cells. Antioxid Redox Signal. 2000 Winter; 2 (4):789-99). Studies by Han et al show that ADT is more bioavailable lipid-based formulations, as sub-micro emulsion (SME) and oil solution prepared using short (SCT), medium (MCT) and long (LCT) chain triglycerides respectively. (Han S F, Yao T T, Zhang X X, Gan L, Zhu C, Yu H Z, Gan Y. Int J Pharm. Lipid-based formulations to enhance oral bioavailability of the poorly water-soluble drug anetholtrithione: effects of lipid composition and formulation. 2009 Sep. 8; 379(1):18-24. Epub 2009 Jun. 7.). The emulsion or water soluble compound of ADT ophthalmic drops can be used after insulin drops. Insulin can be combined with the formulation to instill to the eye with one dispenser. The ADT is non toxic. ADT can be very efficacious in treating retinitis pigmentosa associated with or without dry eye syndrome.

### Example 24

Follow the instruction as described in the above EXAMPLE 1. Alagebrium (known as ALT-711) is the first drug to be clinically tested for the purpose of breaking the cross links caused by advanced glycation end products (AGEs), thereby, reversing one of the main mechanisms of aging. This has been seen in diabetics at an early age which glycation may be in retinitis pigmentosa. The drying seen in the diabetics and the aged can be related to AGEs due to carbohydrates binding to proteins including structural proteins, lipids, and DNA.

This process can impair the normal function of organs that depend on flexibility and proper nutrition supply for normal functioning. AGEs cross links leads to loss of function of tissues and induces oxidative stress which AGEs reacts with molecules provokes the underlying component of inflammation. Hence the Alagebrium eye drops in combination with Insulin can prevent AGEs formation, facilitate their removal, and reverse the disease state affecting the photoreceptors function; relive from further development and advancement of retinitis pigmentosa, cataract with diabetic retinopathy.

### Example 25

Follow the instruction as described in the above EXAMPLE 1. There isn't a definitive cure for retinitis pigmentosa. Another objective of our invention is to cure the retinitis pigmentosa cases with no genetic etiology. The genes account for no more than 60% of all patients; the remainder have defects in unidentified genes. Findings of controlled trials indicate that nutritional interventions, including vitamin A palmitate and omega-3-rich fish, slow the progression of the disease in many patients. The findings indicate that our invention with the use of Insulin, where these nutritional supplements can arrest and can cure about 40% of the patients, whom don't show the genetic based photoreceptors apoptosis leading to retinitis pigmentosa.

### Example 26

Follow the instruction as described in the above EXAMPLE 1. Oral intake of Vitamin A, B₆, C, D₃, E, GLA, has been known to delay the progression of the retinitis pigmentosa. Vitamin E seems to play a role which it works together with vitamins A and D. Vitamin D is the only molecule that we create ourselves from light and turn into a hormone (OH25D). An amazing feat when you think about the process. Similarly, Vitamin A, obtained through the diet, is the other dietary lipid-based nutrient, that we turn into a hormone (retinoic acid) to be used by the photoreceptors pigment formation for light rectption. These supplements will help the condition of retinitis pigmentosa. Insulin drops should be used 30 minutes to one hour after taking these supplements orally to enhance their uptake by the disease afflicted cells. In the eyes, these supplements circulates through the choroidal BV and are transported through the RPE to the outer segment of the photoreceptors.

The progression of the disease can be reduced by the daily intake of 15000 IU (equivalent to 4.5 mg) of vitamin A palmitate. Eleven-CIS Vitamin A can be used for treating this condition (Berson E L, Rosner B, Sandberg M A, et al. (1993). “A randomized trial of vitamin A and vitamin E supplementation for retinitis pigmentosa”. Arch. Opthalmol. 111 (6): 761-72). Recent studies have shown that the vitamin A supplementation can postpone blindness by almost 10 years (Berson E L (2007). “Long-term visual prognosis in patients with retinitis pigmentosa: the Ludwig von Sallmann lecture”. Exp. Eye Res. 85 (1): 7-14). Scientists continue to investigate possible treatments with less success. Vitamin A deficiency is more common than we realize resulting in malfunction of the photoreceptors. The vitamin A—rich foods are rarely eaten which the vitamin A toxicity has been overblown to our profound immunological detriment. Vitamin A is necessary for optimal mucosal immunity. The vitamin A is needed for the formation of photoreceptors pigment which the pigment is needed for vision.

Vitamin A is a key nutrient in balancing the newly discovered pro-inflammatory cytokine, IL-17. Carotenes aren't an adequate or safe substitute for vitamin A in supplements in retinitis pigmentosa. Carotenes and carotene rich foods such as sweet potatoes, carrots, kale, spinach, turnip greens, winter squash, collard greens, cilantro, fresh thyme, cantaloupe, romaine lettuce and broccoli; have long been recommended and promoted as a substitute. New research shows that the carotenes aren't efficiently converted to vitamin A in as many as 50% of the individuals. The carotenes can create cleavage products, which the products form free radicals, that these radicals interrupt vitamin A's protective function. Hence, there is importance to take adequate amounts of Vitamin A where the patient doesn't depend upon its precursor of Carotenes.

Our invention involves taking prescribed amounts of Vitamin A. The patient needs to wait for the vitamin A to be absorbed which the absorption will take about one hour to occur. The blood concentration of vitamin A reaches the peak level at one hour. Then, instill 0.5 to 1.00 units' insulin containing (per drop) in both eyes. The patient should wait 5-10 minutes for the insulin to be absorbed. The absorbed insulin in the retina will enhance the uptake of the circulating vitamin A by photoreceptors where the effect will be therapeutic in curing or curtailing the retinitis pigmentosa. Other vitamins such as Vitamin E, and D3 can be incorporated into Vitamin A ophthalmic drops.

### Example 27

Follow the instruction as described in the above EXAMPLE 1. Scientists at the Osaka Bioscience Institute have identified a protein, named Pikachurin which they believe could lead to a treatment for retinitis pigmentosa (Sato S, Omori Y, Katoh K, et al. (August 2008). “Pikachurin, a dystroglycan ligand, is essential for photoreceptor ribbon synapse formation”. Nat. Neurosci. 11 (8): 923-931). Our invention incorporates pikachurin along with insulin to make more effective in the treatment of retinitis pigmentosa.

### Example 28

Follow the instruction as described in the above EXAMPLE 1. Attempts have been made at University College London Institutes of Opthalmology and Child Health and Moorfields Eye center to treat successfully the induced retinitis pigmentosa with stem cell transplant in mice with retinitis pigmentosa resulting in photoreceptor development with the necessary neural connections. Previously, belief was that the mature retina has no regenerative ability. The use of our invention with insulin ophthalmic drops augments rapid incorporation and differentiation of stem cells into the retina in any stem cell therapy. The insulin allows the stem cells to differentiate the photoreceptors, and the stem cells get connected to other retinal and central neurons.

### Example 29

Follow the instruction as described in the above EXAMPLE 1. Studies involves the use of desmethyldeprenyl, a metabolite of the anti-Parkinson's drug, deprenyl for retinitis pigmentosa (W. A. Baumgartner. Etiology, pathogenesis, and experimental treatment of retinitis pigmentosa. Medical hypothesis. Volume 54, Issue 5, Pages 814-824. May 2000). The rationale is based on an observation that desmethyldeprenyl exerts antiapoptotic activities in a variety of neurodegenerative disorders. The protective mechanism involves the over expression of the anti-apoptotic bcl-2 gene, leading to higher concentrations of bcl-2 proteins, which the proteins binds to mitochondria that the protein inhibits. The trigger mechanism of apoptosis—is the opening of permeability transition pore (PTP), and the release of cytochrome C. At the same time, desmethyldeprenyl causes the underexpression of the pro-apoptotic bax gene which via bax proteins facilitates the opening of the PTP.

Both the anti-apoptotic and pro-apoptotic mechanisms appear to be mediated by the binding of desmethyldeprenyl to glyceraldehyde-3-phosphate dehydrogenase. Antiapoptotic effects can be generated by the parent compound, deprenyl when this is used daily in low concentrations of 1-2 mg/100 kg body weight. These conditions appears that the anti-apoptotic metabolite, desmethyldeprenyl, predominates over the pro-apoptotic metabolites of deprenyl,I-methamphetamine and I-amphetamine. Methamphetamine isn't formed if desmethyldeprenyl is administered directly. The administration could give desmethyldeprenyl a pharmacokinetic advantage over deprenyl. However, desmethyldeprenyl is still an FDA-unapproved substance. The possibility is that deprenyl may have unique anti-apoptotic effects.

The structural similarity to desmethyldeprenyl, cannot be excluded at the present time. Use of availabel deprenyl as ophthalmic drops with or without oral intake with insulin ophthalmic drops can prevent the apoptosis seen in retinitis pigmentosa rod photoreceptors and other oculopathies related to the retina.

### Example 30

Follow the instruction as described in the above EXAMPLE 1. There are patients with retinitis pigmentosa associated with cystoid macular edema. Treatment of this condition is an important part where the treatment of retinitis pigmentosa is to improve the acuity and closer vision. The treatment involves the Intravitreal injection of 4 mg (0.1 ml) triamcinolone acetonide to treat macular edema.

The visual and anatomic responses were observed where there were complications related to the injection procedure and the corticosteroid medication. These patients' eye conditons were treated with 250 mg of oral acetazolamide twice daily for a month or so. Our invention involves using intravitreal injection of triamcinolone acetonide with 1 or 2 units of insulin added to the injectate for its rapid uptake and augmentation-amplification effects. It will make up 0.2 ml injectate which the injectate can be safely injected.

The insulin enhaces the uptake of this corticosteroid, and enhances the corticosteroid acitivity relieving the macular edema at the same time which this activity helps to reduce the ROS causing the damage to the photoreceptors. The use of insulin ophthalmic drops with corticosterioid two to three times a day as part of the protocol for treating retinitis pigmentosa and macular edma with acetazolamide.

### Example 31

Follow the instruction as described in the above EXAMPLE 1. Superoxide dismutases, catalases, lactoperoxidases, glutathione peroxidases and peroxiredoxins, small molecule antioxidants like ascorbic acid (vitamin C), tocopherol (vitamin E), uric acid, polyphenol antioxidants, and glutathione play important roles as cellular antioxidants by facilitating the removal or ROS. The most important plasma antioxidant in humans is probably uric acid. We have used uric acid in many ophthalmic conditions for 30 years. Most of the above antioxidants can be incorporated to ophthalmic drops with insulin. The use of uric acid to prevent and to treat many oculopathies including retinitis pigmentosa.

### Example 32

Follow the instruction as described in the above EXAMPLE 1. Insulin composition with sodium fluorescein combination is used for diagnosing the health and the disease of the eyes' blood supply. The blood supply plays a role in retinitis pigmentosa and diabetic retinopathy. Insulin enhnaces the uptake and the circulation of the eye which the fluorescein will mark the afflicted tissue particularly in the blood vessels and the endothelial cells of the retina. The blood vessels are important for the health of the photoreceptors and the diagnosing underlying patho-physiology related to BV such as in diabetic retinopathy. This diagnostic method called the “fluorangiography” is performed by means of the intravenous injection of a fluorescent substance with the following photography of the retina at different times. Apply ophthalmic muslin drops to both the eyes 30 minutes before the IV injection of fluorescent substance. Insulin can be injected up to 3 units with a fluorescent substance. The fluorescent substance in blood arrives at the retina. The fluorescein colors the BV. This renders the BV visible due to the local effect of the insulin. The results will reveal the functional state of the BV walls. Our invention of insulin ophthalmic solutions can be used to enhance the uptake of radioactive material used to diagnose eye diseases and/or used to treat all eye diseases (RP and Opthalmic tumors).

### Example 33

Follow the instruction as described in the above EXAMPLE 1. Use of Chelation therapeutic agents with insulin: It is a known fact that the photoreceptors in retinitis pigmentosa are undergoing changes and apoptosis due to deposits of fat, calcium, protenacious, and dysfunctional cellular complexes. These changes may take place in the choroid, RPE, Bruch's membrane, photoreceptors, and Muller cells. It is likely that they do have many metallic and organic deposits like the lipoprotenacious material, iron, and calcium, in them due to death of cells and protenacious deposits. Chelation therapy locally or systemically with Ethylenediaminetetraacetic acid (EDTA), Methylsulfonylmethane (MSM), Alagebrium, and Deferoxamine (also known as desferrioxamine B, desferoxamine B, DFO-B, DFOA, DFB or desferal) will clear these clogged cell layers and photoreceptors cells undergoing changes. They remove any excess iron, calcium, and other metals as well as the fatty protenacious deposits which these may interfere with their physiological role.

It is known that the Ethylenediaminetetraacetic acid (EDTA) unclogs blood vessels; controls free radical damage due to lipid peroxidation by serving as a powerful antioxidant; increases tissue flexibility by uncoupling age-related cross-linkages that these are responsible for loss of cellular function; removes lead, cadmium, aluminum, and other metals, restoring enzyme systems to their proper functions; enhances the integrity of cellular and mitochondrial membranes; reduces the tendency of platelets to cause coagulation too readily which the platlets can clog the transportation system; unclogs the clogged draining vascular system, increases tissue flexibility by uncoupling age-related cross-linkages (age-related glycation) which this function is responsible for the proper function of the glands.

Millions of Americans have undergone Chelation therapy including the inventor, to eliminate the arteriosclerotic vascular diseases and to reduce the metalloporoteinases with good results. The inventor has used Chelation therapy with insulin with mild hyperthermia with wonderful results in ASVD. The use of EDTA along with insulin as described in our invention can slow down, arrest, or reverse the changes in the choroidal capillaries, RPE. This brings about the physiological status to the afflicted retinitis pigmentosa rods and cones in the retina.

### Example 34

Follow the instruction as described in the above EXAMPLE 1. Methylsulfonylmethane (MSM), is an organosulfur compound with the formula (CH₃)₂SO₂; a metabolite of DMSO. It is also known by several other names: DMSO2, MSM, Methylsulfonylmethane, methyl sulfone, and dimethyl sulfone. MSM is a supplement form of sulfur that is found in our living tissues. MSM supports healthy connective tissues like tendons, ligaments, muscle, and nervous tissue function including retina. MSM makes cell walls permeable, allowing water and nutrients to freely flow into cells, which the permeability allows the wastes and the toxins to properly flow from the retina, where the outflow is needed in the photoreceptors in retinitis pigmentosa. MSM is an anti-oxidant in which MSM helps to clean the blood-stream.

The MSM flushes toxins trapped in our cells including the photoreceptors. The MSM is a foreign protein and free radical scavenger which the foreign protein is needed to maintain the photoreceptors function affected in retinitis pigmentosa. The body uses MSM along with Vitamin C to create new, healthy cells. The MSM provides the flexible bond between the cells. We have prescribed MSM ophthalmic drops to many aged, Lyme disease, and cancer patients, which the patients reported, that their vision had improved. MSM is soluble in water, and a good solvent like DMSO.

We have used aqueous solutions of MSM filtered, sterilized, and mixed with insulin. We used as eye drops to treat retinitis pigmentosa, dry eye syndrome, glaucoma, and other oculopathies with good results. The use of MSM with insulin as eye drops can prevent (act as prophylactic in those who are genetically disposed), delay the onset, curtail, or cure the retinitis pigmentosa conditions.

We prepare the following eye drops containing: 1. EDTA, 2. Deferoxamine, 3. MSM, with added preservatives, antibacterial, and DMSO combined with insulin in proper concentrations. Any one of the chelating agents or combination of them can be used to formulate the eye drops. These eye drops are used before or after insulin drops as prophylactic and therapeutic agents for retinitis pigmentosa and other oculopathies.

### Example 35

U.S. Patent Application Publication Number: 2004/0054130 A1 invention relates to compounds, which the compounds have the ability to potentiate the physiological activity of insulin particular to the small peptide compounds or peptidomimetic compounds, where the compounds has the ability to potentiate one or more of the physiological activities of insulin. The peptides comprises a basic amino acids, such as lysine, arginine, homolysine, homoarginine or ornithine; neutral aliphatic amino acid, in either the L—or the D—form, such as glycine, leucine, alanine, phenylalanine or isoleucine, homo leucine, norleucine, homonorieucine, cyclohexylalanine, or homocyclohexylalanine; an aromatic amino acid, such as phenylalanine or tyrosine. The amino acids or amino acid analogues have a side chain having or delocalized electrons.

Any one of these therapeutic agents can be added to the ophthalmic preparations of the insulin to enhance the insulin absorption and the activity to treat retinitis pigmentosa and other oculopathies.

### Example 36

Follow the instruction as described in the above EXAMPLE 1. If the corneal, conjunctival and retinal BV are supected of involved in oculopatheis, they need to be tested using fluorescein as one of the method testing before treating retinitis pigmentosa. The fluorangiography is performed by means of the intravenous injection of a fluorescent substance with following photography of the retina and the retinas' BV at different times. The fluorescent substance in blood arrives at the retina which the substance colors the arteries, the capillaries, and the veins. The fluorescent substance renders BV visible, with the functional state of their walls. Use of our invention with insulin before the procedure or with IV injection of the dye demarks the afflicted blood vessels even better.

Any thinning of the retinal blood vessels and associated ocular pathology is revealed by this method. Local use of this fluorescent sustances to diagnose corneal and conjunctival pathology can be facilitated using a mixture of the dye and muslin or using ophthalmic insulin drops before instilling the marker dyes.

### Example 37

Follow the instruction as described in the above EXAMPLE 1. U.S. Patent Application Publication Number: 2003/0065020 A1 describes a method of treating or preventing macular degeneration in patients by administering HMG-CoA reductase inhibitors. This invention discloses the treatment with HMG-CoA reductase inhibitors results in: (i) reduced accumulation of basal linear deposit in Bruch's membrane; (ii) protection of the outer retina from oxidative damage; and (iii) inhibition of endothelial cell apoptosis.

Oral intake HMG-CoA reductase inhibitors can be used to treat RP to prevent the oxidative damage, clear the linear fatty deposits in the Bruch's membrane so that it can actively participate in the photoreceptors physiological function, and prevent photoreceptors apoptosis seen in this condition. There are various HMG-CoA reductase inhibitors in use and it is selected from the group consisting of: fluvastatin (Lescol), cerivastatin (Baycol), atorvastatin (Lipitor), simvastatin (Zocor), pravastatin (Pravachol), lovastatin (Mevacor) and rosuvastatin (ZD 4522). We prepare suitable ophthalmic drops from one of these statins for use with insulin ophthalmic drops. Alternatively, use the statins orally with insulin drops to inhibit the pathological process in the retina. We recommended the statins drugs in varying doses to almost all the patients with these conditions including diabetic retinopathy and age related macular degeneration. This method of therapy not only saves the photoreceptors degradation in this disease, it also saves the patients from the cardiovascular diseases.

Numerous modifications; alternative arrangements of steps explained and examples given herein may be devised by those skilled in the art without departing from the spirit and the scope of the present invention. The appended claims are intended to cover such modifications and arrangements. Thus, the present invention has been described above with particularity and detail in connection. This is presently deemed to be the most practical and preferred embodiments of the invention. The invention will be apparent to those of ordinary skill in the art that numerous modifications, including, but not limited to, variations in size, materials, shape, form function, and manner of procedure, assembly, and the use may be made. The preferred embodiment of the present invention has been described. The invention should be understood that various changes, adaptations, and modifications may be made thereto. It should be understood, therefore, that the invention is not limited to details of the illustrated invention. This method can be used to diagnose corneal ulcers; any pathological changes in the cornea and conjunctiva of the eye.

The preferred embodiment of the present invention has been described. The invention should be understood that various changes, adaptations and modifications may be made thereto. This should be understood, therefore, that the invention is not limited to details of the illustrated invention examples.

What is claimed is:

1. A method of treating retinitis pigmentosa comprising the step of topically instilling a therapeutically effective dose of insulin to a retinitis pigmentosa afflicted eye's conjunctival sac in humans and animals to be delivered to afflicted photoreceptors and the retina.

2. The method of treating retinitis pigmentosa according to claim 1 further comprising the step of instilling at least one additional therapeutic, pharmaceutical, biochemical, and biological agents or compounds to said afflicted eye.

3. A method of treating retinitis pigmentosa comprising the step of topically instilling a therapeutically effective dose of IGF-1 to a retinitis pigmentosa afflicted eye conjunctival sac to be delivered to the afflicted photoreceptors and the rest of the retina.

4. A method of treating retinitis pigmentosa comprising the step of topically instilling a therapeutically effective dose of insulin and IGF—with combination to the afflicted eye conjunctival sac to be delivered to the afflicted photoreceptors and retina.

5. The method of treating retinitis pigmentosa according to claim 3 further comprising the step of applying at least one other therapeutic, pharmaceutical, biochemical, and biological agents or compounds to said afflicted eye to be delivered to the afflicted photoreceptors and the retina.

6. The method of treating retinitis pigmentosa according to claim 2 wherein said therapeutic agent is a pharmaceutical agent selected from the group consisting of biochemical, organic, and inorganic agents.

7. The method of treating retinitis pigmentosa according to claim 2 wherein said known therapeutic agent is selected from a group consisting of cyclosporins in aqueous base or oily base.

8. The method of treating retinitis pigmentosa according to claim 2 wherein said known therapeutic agent is a biological agent selected from the group consisting of Monoclonal Antibodies Remicade™, Etanercept, Embrel™, and Humira™, TNF anti TNF agents, agents targeting TNF-α and B cells, anti-CD20 and anti-CD22.

9. The method of treating retinitis pigmentosa according to claim 2 wherein said known therapeutic agent is selected from the group consisting of testosterone; DHEA, estrogens; Hydroxychloroquine (Plaquenil) and azathioprine (Imuran).

10. The method of treating retinitis pigmentosa according to claim wherein said known therapeutic agents are ophthalmic preparations selected from the group consisting of Anetholdithiol thione (ADT, 5-[p-methoxyphenyl]-3H-1,2-dithiol-3-thione); pilocarpine (Salagen), and cevimeline (Evoxac).

11. The method of treating retinitis pigmentosa according to claim wherein said known therapeutic agent is a combination of at least two agents selected from the group consisting of cyclosporins, estrogens, DHEA, and testosterone.

12. The method of treating retinitis pigmentosa according to claim 2 wherein known therapeutic agent is Curcumin.

13. The method of treating retinitis pigmentosa according to claim wherein said known therapeutic agent is selected from the group consisting of hyaluronic acid, Diquafosol (INS365 Ophthalmic) and Rebamipide.

14. The method of treating retinitis pigmentosa according to claim wherein said known therapeutic agent is a chelating agent selected from the group consisting of Methylsulfonylmethane (MSM), Ethylenediaminetetraacetic acid (EDTA), Alagebrium and Deferoxamine.

15. The method of treating retinitis pigmentosa according to claim 2 further comprising the step of using an uptake facilitator to further enhance the therapeutic effect selected from the group comprising electroporation, iontophoresis, sonophoresis, vibroacoustic, vibration, physical heat, magnetic field, radio frequency field, microwave, and laser light.

16. The method of treating retinitis pigmentosa according to claim wherein said known therapeutic agents are selected from the group consisting of Vitamin A, C, E, B6, phospholipids, liposome-based; retinoids; glycerin, propylene glycol, glutathione, uric acid, polyphenol antioxidants, Resveratol, superoxide dismutase's, catalases, lactoperoxidases, glutathione peroxidases, peroxiredoxins, and calcium ion ophthalmic drops compositions.

17. The method of treating retinitis pigmentosa according to claim wherein said known therapeutic agents are selected from the group consisting of antibiotics, analgesics, and antivirals.

18. The method of treating retinitis pigmentosa according to claim wherein said known therapeutic agents are selected from the group consisting of Gamma linolenic acid and omega 3 fatty acid (DHA and EPA).

19. The method of treating retinitis pigmentosa according to claim 18 further comprising the step of combining said known therapeutic agent with at least one of vitamin A and vitamin E.

20. The method of treating retinitis pigmentosa according to claim 2 wherein said known therapeutic agents are selected from the group consisting of antihistamines, vasoconstrictors, non-steroidal anti-inflammatories; and corticosteroids.

21. The method of treating retinitis pigmentosa according to claim 2 wherein said known therapeutic agents are acetazolamide and Brinzolamide.

22. The method of treating retinitis pigmentosa according to claim 2 wherein said known therapeutic agents or compounds are selected from the group being adapted to have the ability to potentiate the one or more of the physiological activities of insulin that the peptide comprises a basic amino acid lysine, arginine, homolysine, homoarginine or ornithine; L—or D—form of neutral aliphatic amino acid, glycine, leucine, alanine, phenylalanine or isoleucine, homo leucine, norleucine, homonorieucine, cyclohexylalanine, or homocyclohexylalanine; an aromatic amino acid, phenylalanine or tyrosine.

22. The method of treating retinitis pigmentosa according to claim 2 wherein the known therapeutic agents or compounds are selected from the group consisting of HMG-CoA reductase inhibitor comprising fluvastatin (Lescol), cerivastatin (Baycol), atorvastatin (Lipitor), simvastatin (Zocor), pravastatin (Pravachol), lovastatin (Mevacor) and rosuvastatin (ZD 4522) given orally and as ophthalmic preparation with insulin.
